(19) United States
(12) Patent Application Publication
(10) Pub. No.: US 2021/0379088 A1
(43) Pub. Date: Dec. 9, 2021

(54) CANCER REGRESSION BY INDUCING A REGENERATION-LIKE RESPONSE

(71) Applicants: VIB VZW, Gent (BE); KATHOLIEKE UNIVERSITEIT LEUVEN, K.U.LEUVEN R&D, Leuven (BE)

(72) Inventors: Georg Halder, Herent (BE); Iván Moya, Leuven (BE)

(21) Appl. No.: 17/299,211

(22) PCT Filed: Dec. 3, 2019
(86) PCT No.: PCT/EP19/83485

§ 371 Date: Jun. 2, 2021

(30) Foreign Application Priority Data
Dec. 3, 2018 (GB) 1819659.2

(51) Int. Cl. A61K 31/661; A61K 31/473; A61K 38/17; A61K 38/19; A61K 45/06; A61P 35/00

(52) U.S. Cl. CPC A61K 31/661; A61K 31/473; A61K 38/177; A61K 48/00; A61K 45/06; A61P 35/00; A61K 38/193

(57) ABSTRACT

The invention relates to the field of oncology, in particular to the field of anti-cancer agents or mechanisms. In particular, activation of a regeneration-like response, such as by activating expression and/or function of YAP and/or TAZ in an organ carrying a tumor or cancer is capable of causing regression of that tumor or cancer.

### CROSS-REFERENCE TO RELATED APPLICATIONS

This application is a national phase entry under 35 U.S.C. § 371 of International Patent Application PCT/EP2019/083485, filed Dec. 3, 2019, designating the United States of America and published in English as International Patent Publication WO 2020/115039 on Jun. 11, 2020, which claims the benefit under Article 8 of the Patent Cooperation Treaty to United Kingdom Patent Application Serial No. 1819659.2, filed Dec. 3, 2018, the entireties of which are hereby incorporated by reference.

### FIELD OF THE INVENTION

The invention relates to the field of oncology, in particular to the field of anti-cancer agents or mechanisms. In particular, activation of a regeneration-like response, such as by activating expression and/or function of YAP and/or TAZ in an organ carrying a tumor or cancer is capable of causing regression of that tumor or cancer.

### BACKGROUND

Tumors not only comprise tumor cells but many other, genetically normal stromal cells that are recruited into growing tumors. These include endothelial cells that build new blood vessels, fibroblasts that produce extracellular matrix, and various types of immune cells that can have tumor promoting or tumor suppressing effects (Quail & Joyce 2013, Nat Med 19:1423-1437; Shi et al. 2017, Nat Rev Drug Discov 16:35-52; Ungefroren et al. 2011, Cell Commun Signal 9:18). Tumors are thus complex structures where reciprocal signaling between different cell types is essential for its growth and survival. Indeed, much effort is directed to deciphering the complex interdependencies between tumor cells and cells of the tumor microenvironment with the hope to identify novel vulnerabilities that could be targeted for cancer therapy (Ungefroren et al. 2011, Cell Commun Signal 9:18). These efforts have been highly informative and resulted in the invention of immunotherapy and anti-angiogenic therapy that target stromal cells in order to attack cancer (Bergers & Hanahan 2008, Nat Rev Cancer 8:592-603; Khalil et al. 2016, Nat Rev Clin Oncol 13:273-290). However, while the tumor microenvironment has been vigorously studied, less attention was focused on the interactions between tumors and their normal surrounding “host” tissue. Thus, only little is known about how tumors interact with their host organ, how the tumor-surrounding tissue reacts to the presence of a tumor, and how this reaction affects tumor growth.

In his review, Pan (2010, Dev Cell 19:491-505; and references cited therein) summarized the available knowledge on the Hippo-pathway originally discovered in Drosophila and later found to be conserved (albeit with divergences) in mammals. Transgenic and knockout mouse experiments revealed that overexpression of YAP (mammalian homolog of Drosophila Yorkie, Yki, a transcriptional coactivator regulated by the Hippo signaling pathway; a mammalian homolog of YAP is TAZ), knockout of Mst1/2 (mammalian homologs of Drosophila Hippo, Hpo, kinase), knockout of Savi (mammalian homolog of Drosophila Salvador, Sav, a regulator of Hippo) each led to expansion of liver size, and ultimately, to the induction of hepatocellular carcinoma (HCC). Inactivation of Nf2/Merlin (upstream of Hippo) also led to HCC and bile duct tumors, the latter also observed upon Mst1/2 or Savi knockout.

Amplification of the YAP gene locus is observed in several cancers (medulloblastoma, oral squamous-cell carcinoma, and lung-, pancreas-, oesophagus-, liver-, and breast carcinomas). YAP overexpression is frequently observed in lung, ovarian, pancreatic, colorectal, hepatocellular and prostate cancer, and is a prognostic marker in HCC.

One emerging cancer pathway thus is the Hippo pathway (Harvey et al. 2013; Zanconato et al. 2016). The effectors of this pathway, the YAP and TAZ transcriptional co-activators, regulate gene expression when bound to TEAD family and other transcription factors (Meng et al. 2016). Their activity is regulated by a kinase cascade that comprises the mammalian Ste20-like kinases 1/2 (MST1/2) and the Large tumor suppressor kinases 1/2 (LATS1/2), which phosphorylate YAP/TAZ. Phosophorylation of YAP and/or TAZ inhibits their transcriptional activity and promotes their nuclear export and proteasomal degradation. When the LATS1/2 kinases are inactive, YAP and TAZ accumulate in the nucleus and promote cell proliferation, stemness, and cell survival (Zanconato et al. 2016). This can lead to the expansion of progenitor cell populations and eventually cancer initiation (Johnson & Halder 2014; Zanconato et al. 2016). The activation of YAP orTAZ actually promotes several hallmarks of cancer cells, such as stemness, cell cycle progression, drug resistance, and increased metastatic potential. YAP and TAZ are thus considered attractive targets for cancer therapy because (i) most human cancers show upregulation of YAP/TAZ levels and/or activity; (ii) downregulation of YAP/TAZ slows the proliferation of cancer cells and tumor growth in vivo in genetic or xenotransplant mouse models and (iii) they are largely dispensable for the normal homeostasis of many adult mouse tissues (Harvey et al. 2013; Johnson & Halder 2014; Zanconato et al. 2016). Accordingly, several ambitious academic and industrial programs aiming at identifying YAP/TAZ pharmacological inhibitors are ongoing. Some results of these programs are discussed hereinafter in the framework of the current invention.

### SUMMARY OF THE INVENTION

In one aspect, the invention relates to enhancers or activators of expression and/or function of YAP and/or TAZ for use in treating or inhibiting cancer, for use in inhibiting progression of tumor growth, or for use in treating or inhibiting tumor metastasis.

The effect of the enhancers or activators on the function and/or expression of YAP and/or TAZ is either direct or is indirect.

The enhancer or activator of expression and/or function of YAP and/or TAZ can be a pharmacologic compound or a gene therapeutic compound. In particular, the enhancer or activator of expression and/or function of YAP and/or TAZ is a nucleic acid capable of activating expression and/or function of YAP and/or TAZ, or is a nucleic acid capable of blocking inactivation of expression and/or function of YAP and/or TAZ.

In any of the above, the enhancer or activator of expression and/or function of YAP and/or TAZ is acting transiently or is inducible, or the blocking of inactivation of expression and/or function of YAP and/or TAZ is transient or inducible.

In any of the above, the enhancer or activator of expression and/or function of YAP and/or TAZ is a nucleic acid capable of driving expression of YAP or of a constitutively active YAP variant; a nucleic acid capable of driving expression of TAZ or of a constitutively active TAZ variant; a nucleic acid capable of driving expression of any combination of YAP, TAZ, constitutively active YAP variant, or constitutively active TAZ variant; or any combination of nucleic acids each individually capable of driving expression of YAP, TAZ, constitutively active YAP variant, or constitutively active TAZ variant. Herein, the enhancer or activator of expression and/or function of YAP and/or TAZ may further be combined, on a same or separate nucleic acid, with a gene capable of driving expression of a TEAD transcription factor.

In any of the above, the enhancer or activator of expression and/or function of YAP and/or TAZ is, or is combined with, a glucocorticoid, sphingosine-1-phosphate (S1P), dihydro-SiP, lysophosphatidic acid (LPA), or ethacridine.

In any of the above, the enhancer or activator of expression and/or function of YAP and/or TAZ is administered locally to an organ having a cancer or tumor, or is peritumoral, peripheral, or systemic; or is for use in administration locally to an organ having a cancer or tumor, or for use in peritumoral, peripheral, or systemic administration.

In any of the above, the enhancer or activator of expression and/or function of YAP and/or TAZ can be administered in conjunction with macrophage colony-stimulating factor 1 (CSF1), beta-catenin, granulocyte colony-stimulating factor (GCSF), a RAGE-inhibitor, or in conjunction with any combination thereof. Alternatively, the enhancer or activator of expression and/or function of YAP and/or TAZ is for use in administration in conjunction with administration of macrophage colony-stimulating factor 1 (CSF1), beta-catenin, granulocyte colony-stimulating factor (GCSF), a RAGE-inhibitor, or in conjunction with any combination thereof.

In any of the above, the enhancer or activator of expression and/or function of YAP and/or TAZ can be administered in conjunction with an attenuator of cell division, an antifibrotic agent, or in conjunction with any combination thereof. Alternatively, the enhancer or activator of expression and/or function of YAP and/or TAZ is for use in administration in conjunction with administration of an attenuator of cell division, an antifibrotic agent, or in conjunction with any combination thereof.

In any of the above, the enhancer or activator of expression and/or function of YAP and/or TAZ can be combined in any way with a further anticancer treatment or antitumor agent. Alternatively, the enhancer or activator of expression and/or function of YAP and/or TAZ is for use in administration in conjunction with a further anticancer treatment or antitumor agent.

Furthermore, the enhancer or activator of expression and/or function of YAP and/or TAZ can be for use in treating or inhibiting cancer, for use in inhibiting progression of tumor growth, or for use in treating or inhibiting tumor metastasis in particular prior to surgical resection of remaining tumor or cancer tissue Furthermore, the enhancer or activator of expression and/or function of YAP and/or TAZ can be for use in treating or inhibiting cancer, for use in inhibiting progression of tumor growth, or for use in treating or inhibiting tumor metastasis in particular prior to administration of an inhibitor of expression and/or function of YAP and/or TAZ.

In any of the above, said cancer or tumor in particular is a liver cancer or is a liver tumor. More in particular, the liver tumor can be liver cholangiocarcinoma, hepatocellular carcinoma or can be a metastatic liver tumor.

In a further aspect, the invention relates to enhancers or activators of liver regeneration for use in treating or inhibiting liver cancer, for use in inhibiting progression of liver tumor growth, or for use in treating or inhibiting liver tumor metastasis.

### DESCRIPTION TO THE FIGURES

FIGS. 1A-1I. Tumor cell survival depends on YAP/TAZ levels in surrounding liver tissue

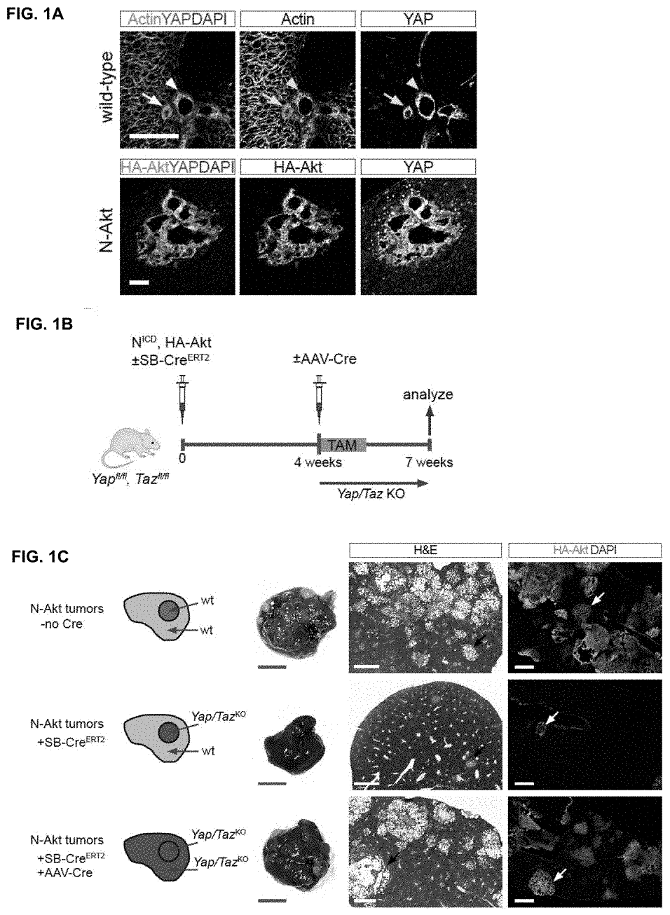

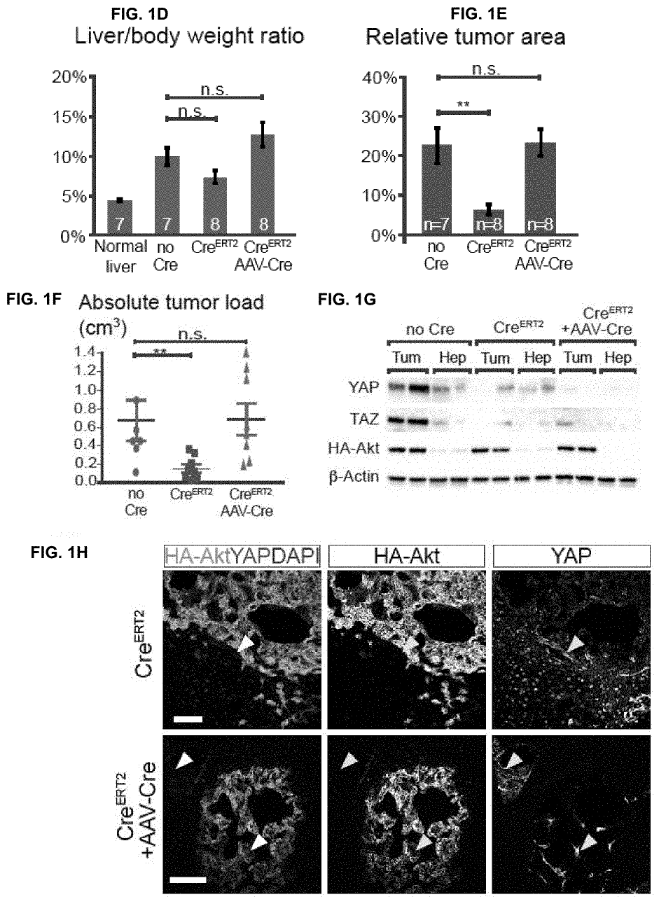

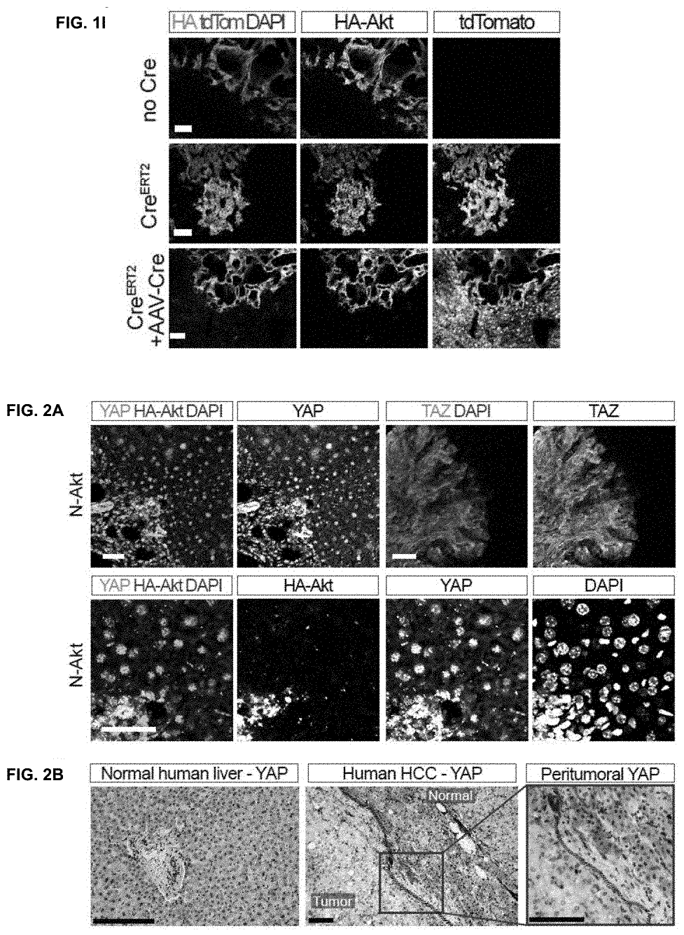

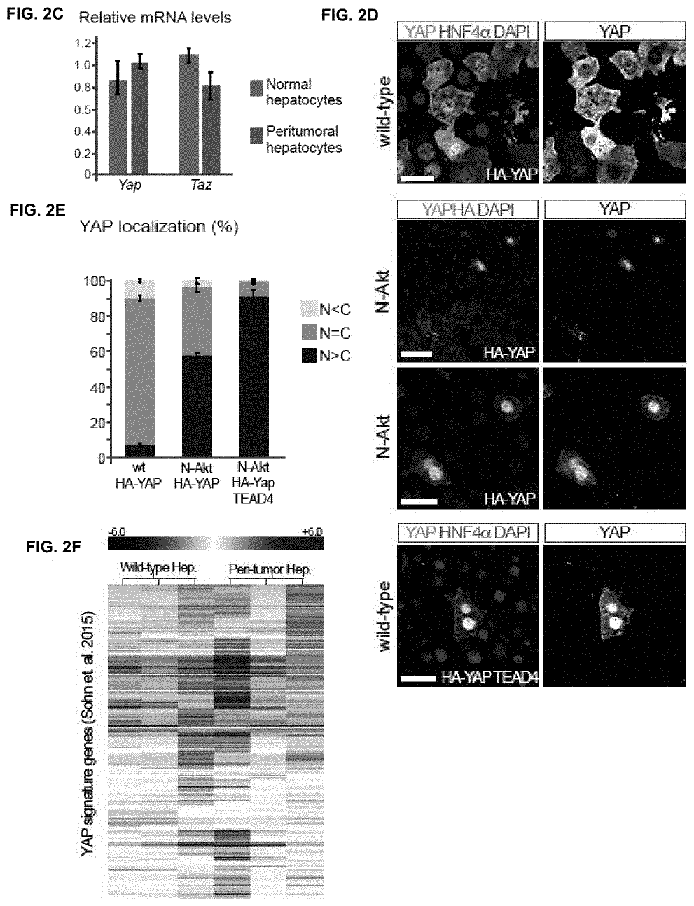

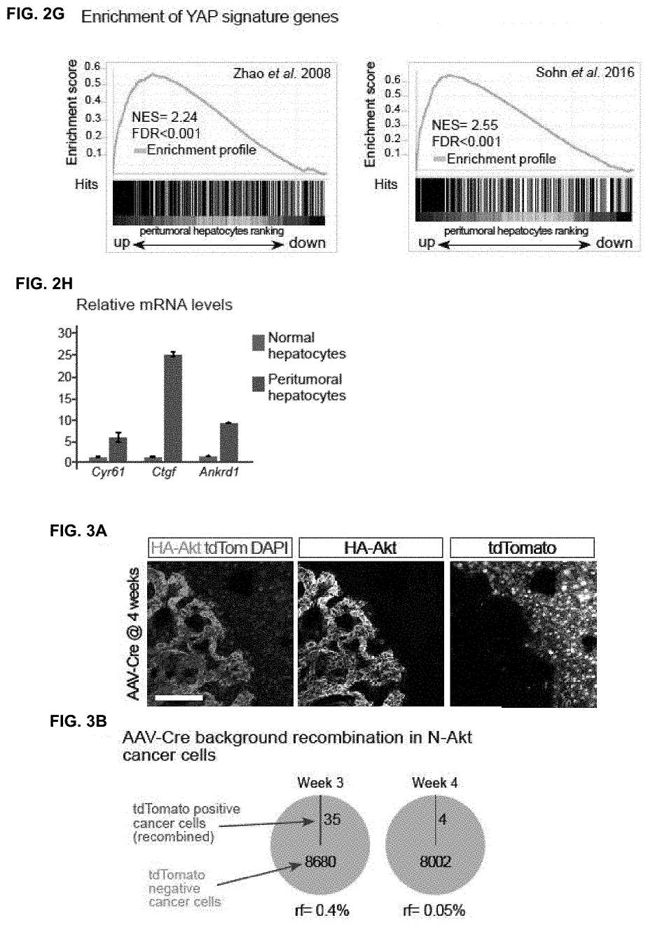

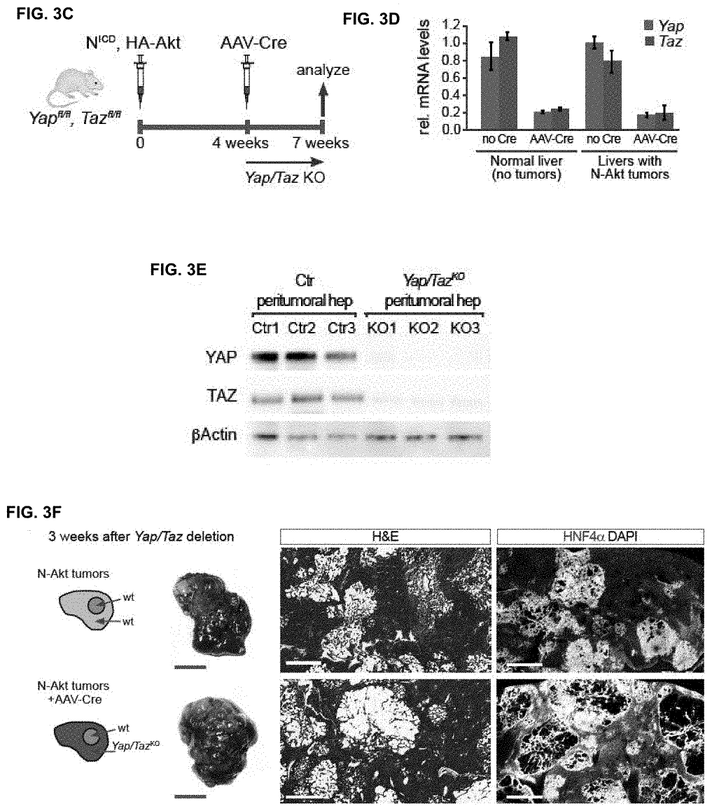

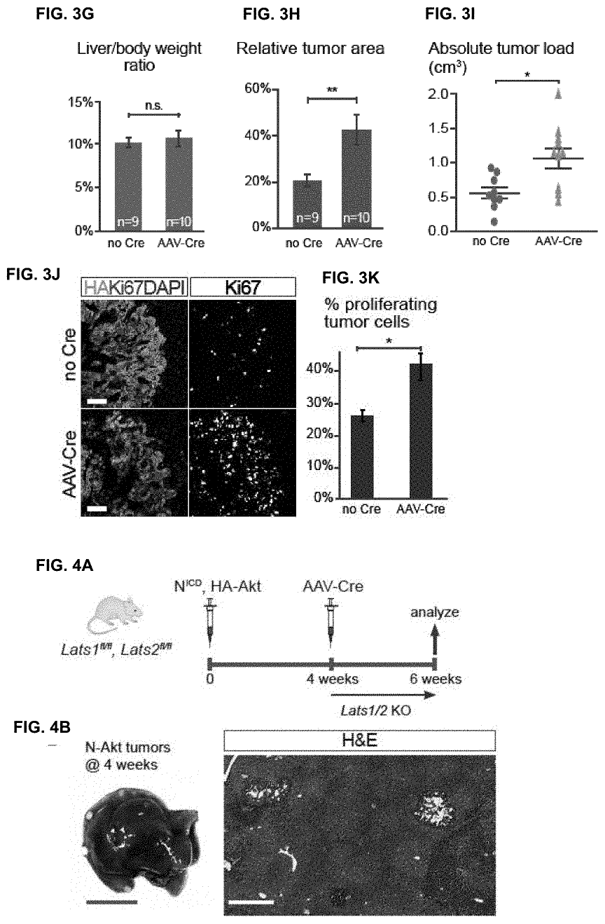

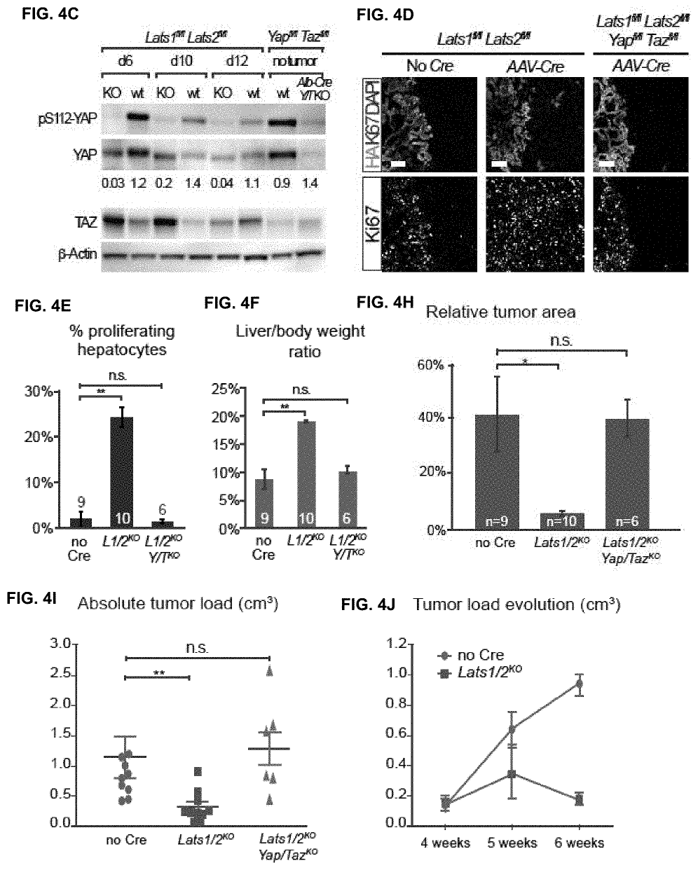

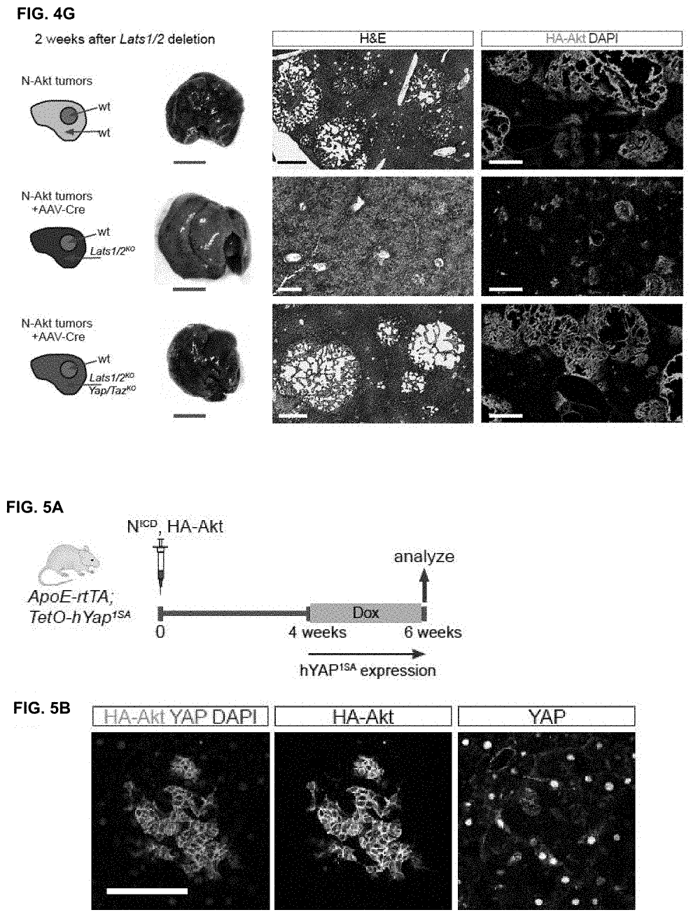

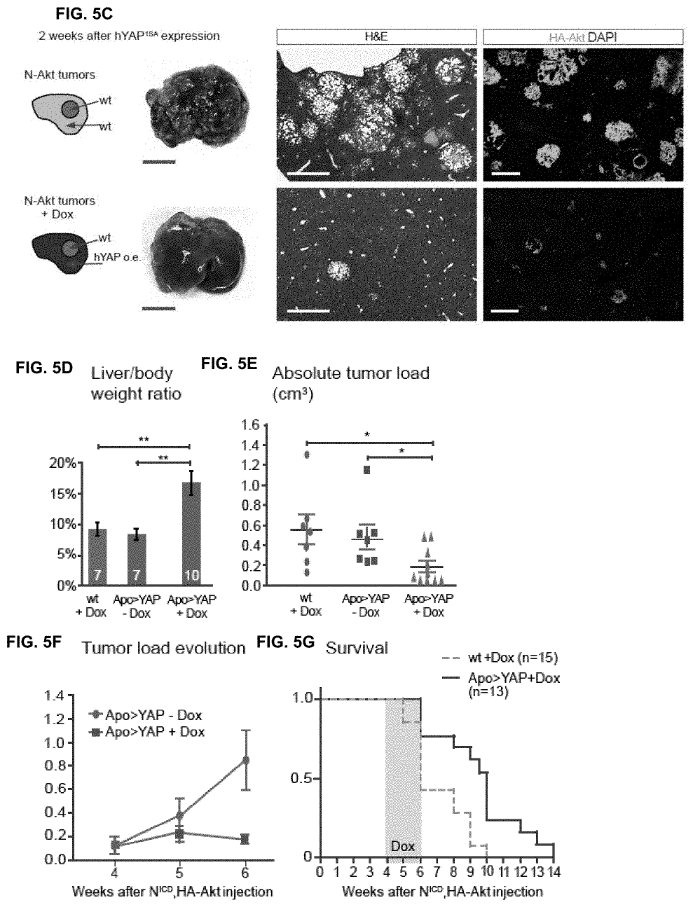

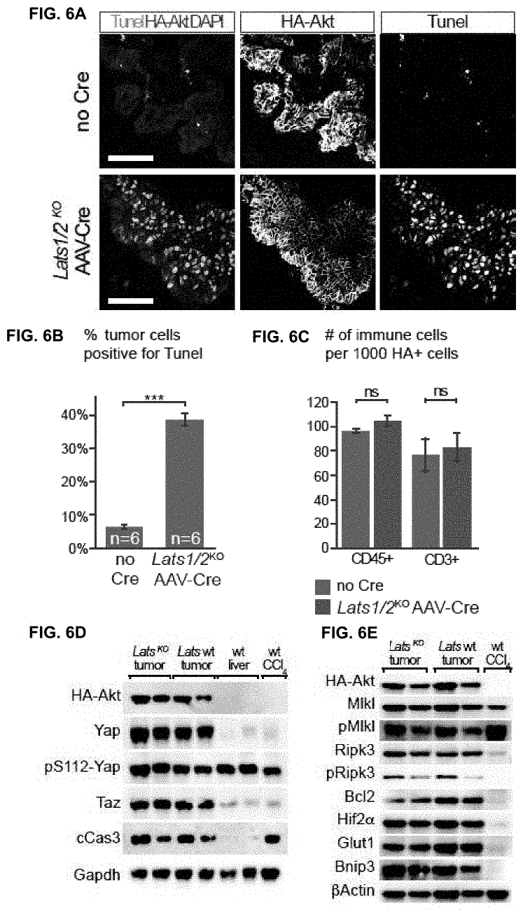

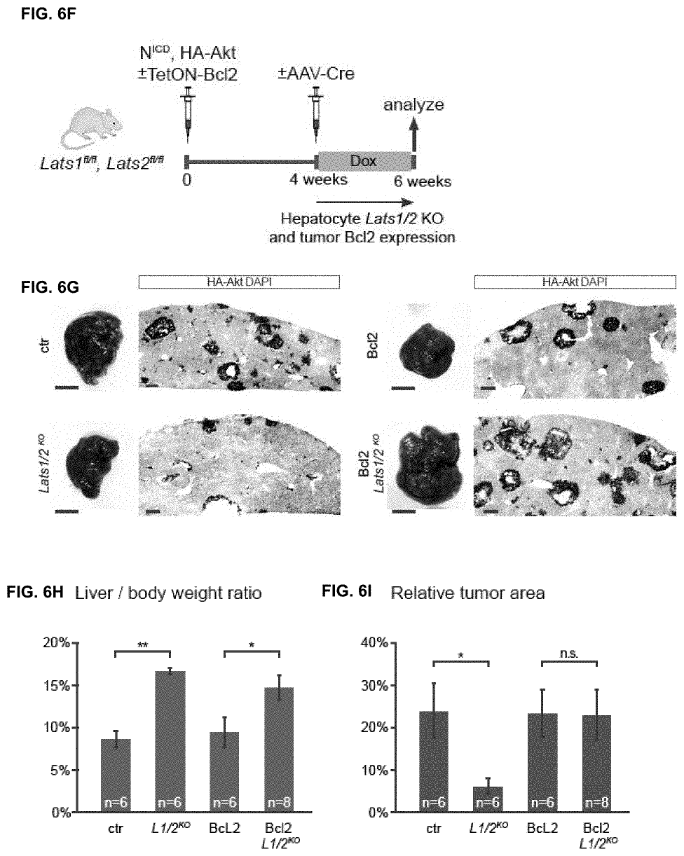

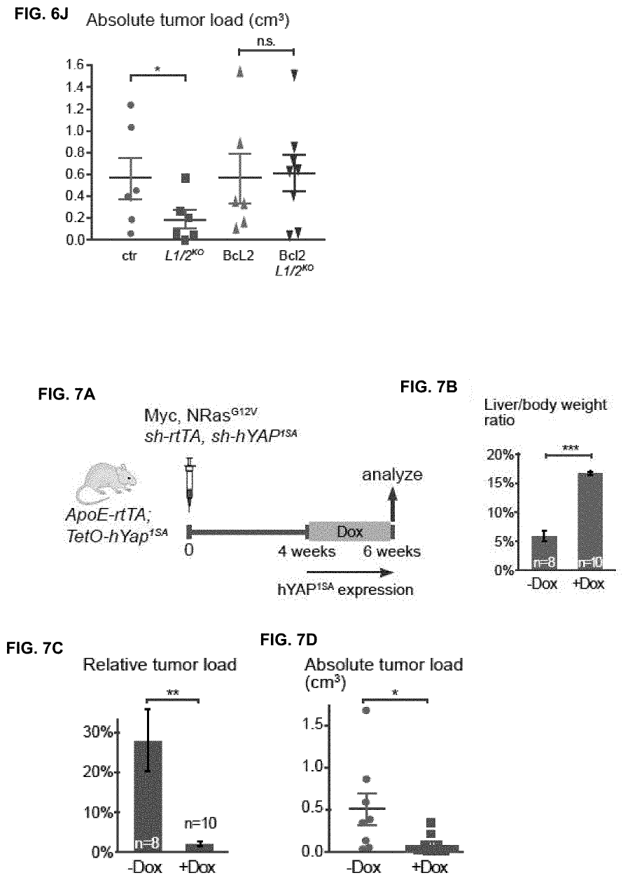

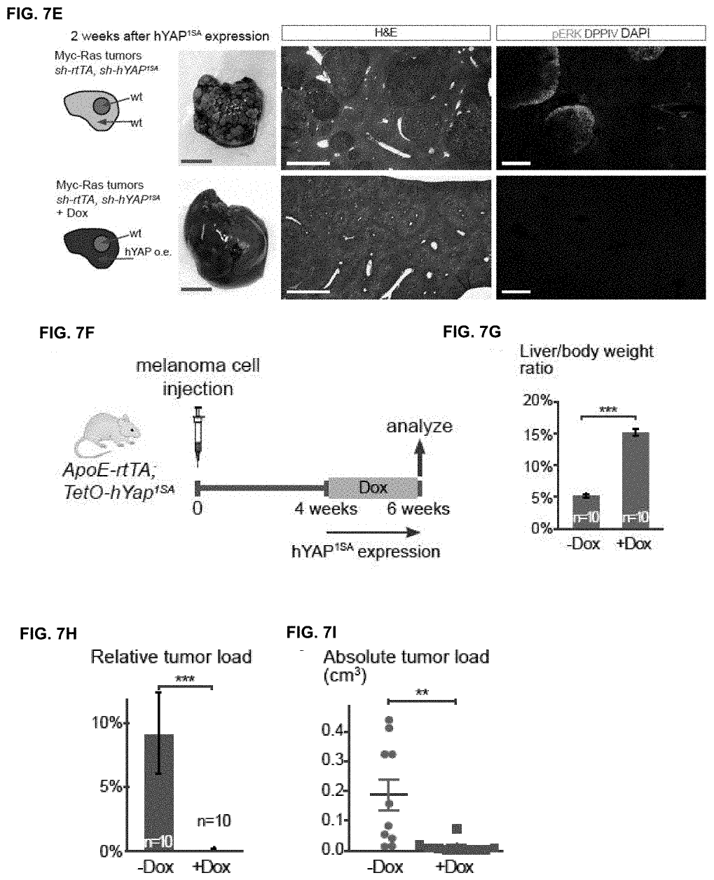

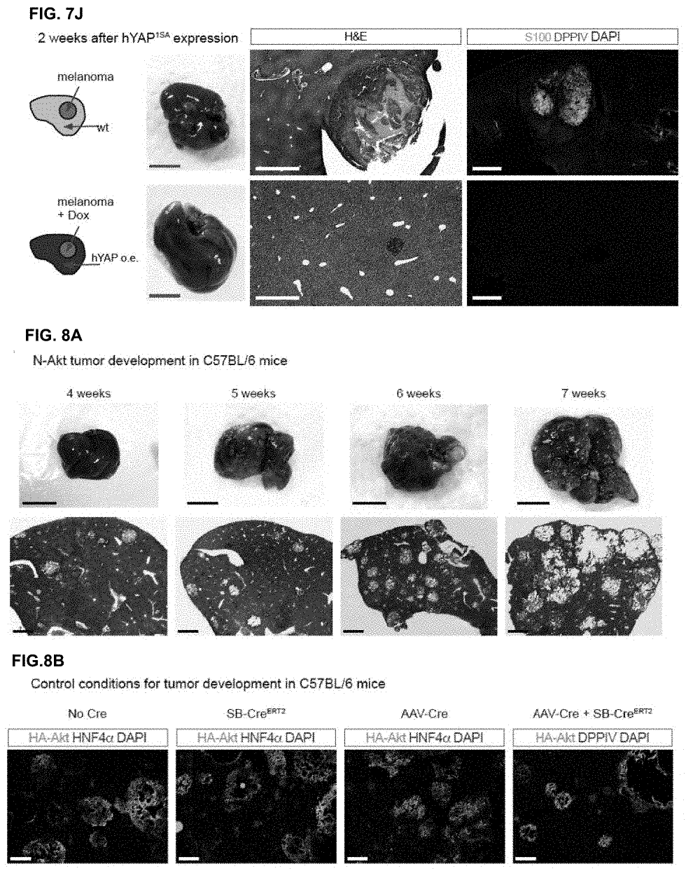

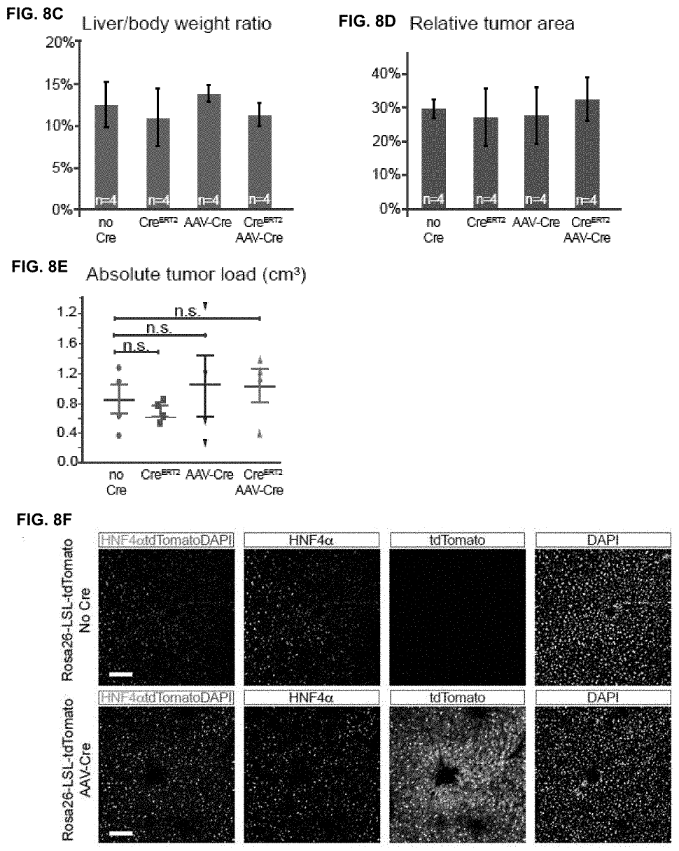

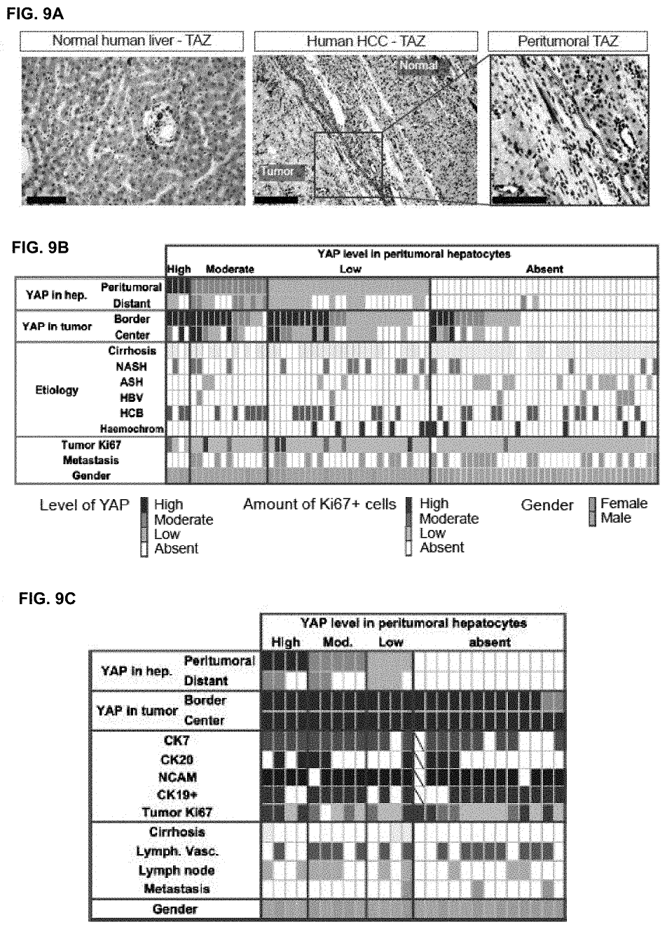

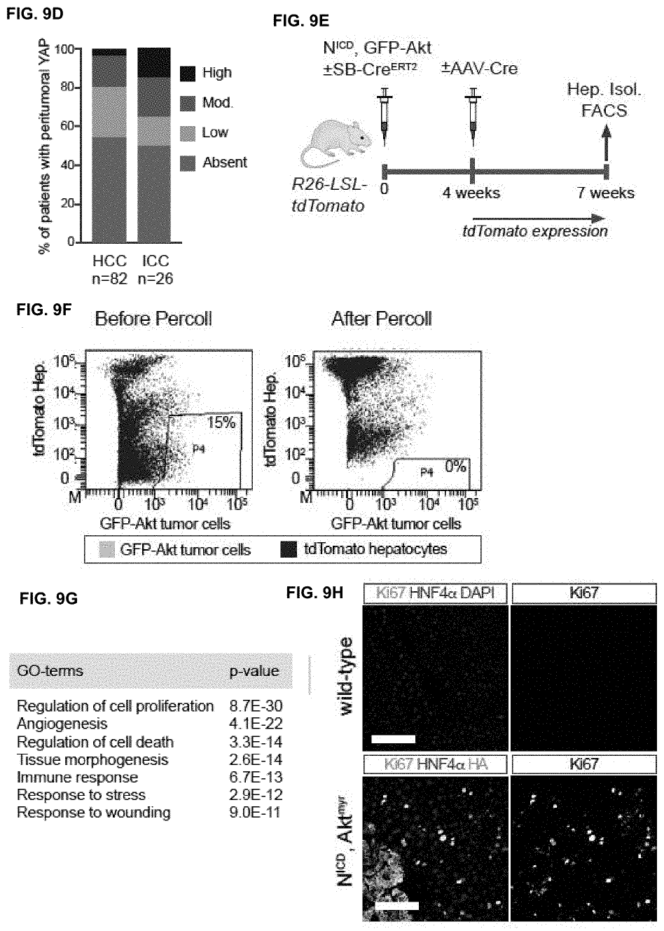

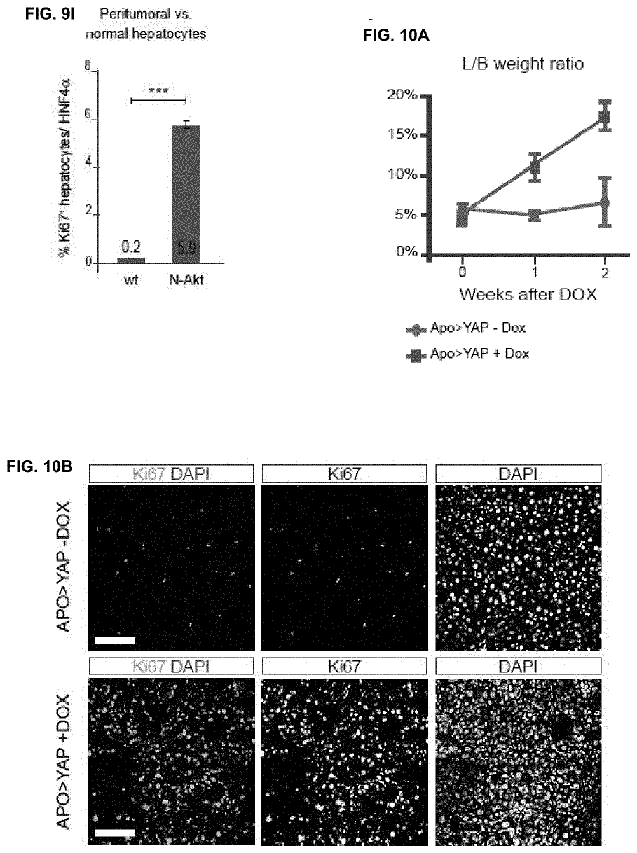

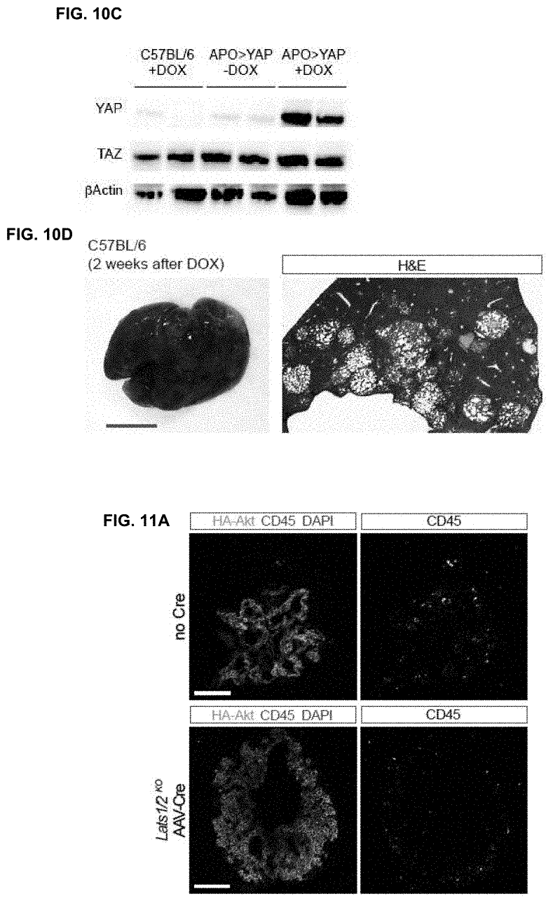

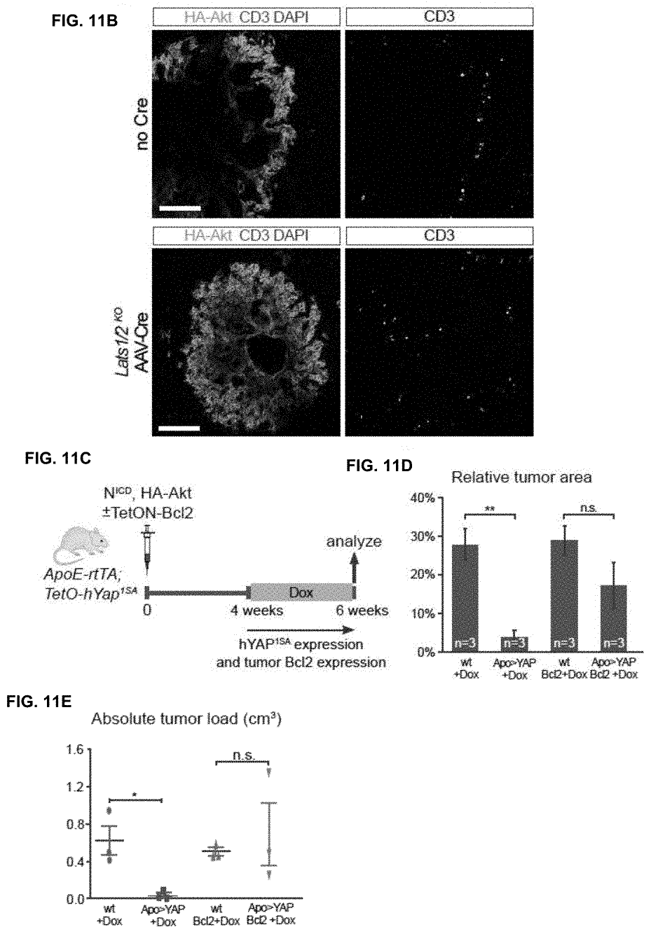

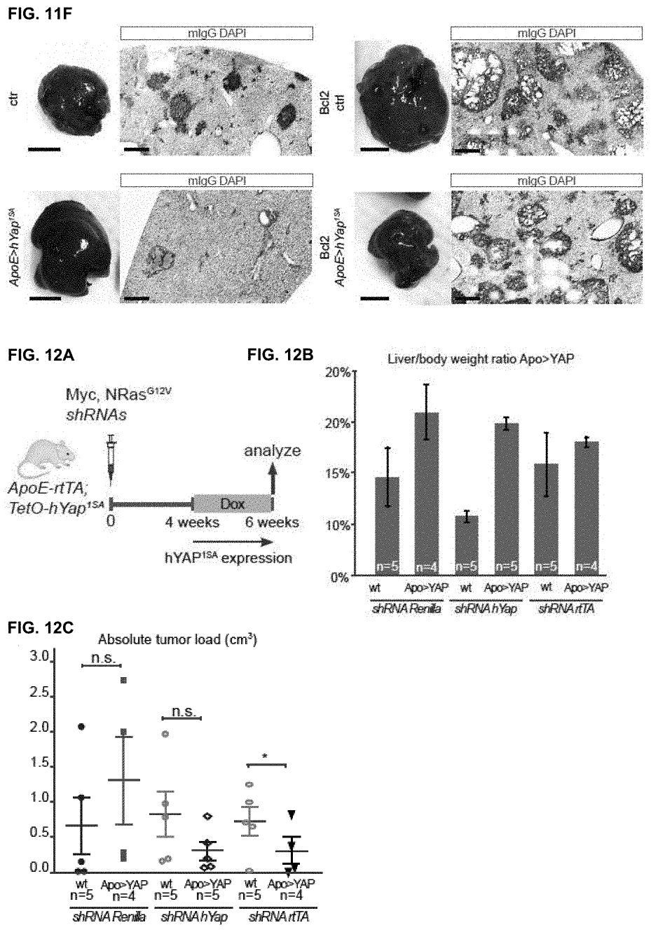

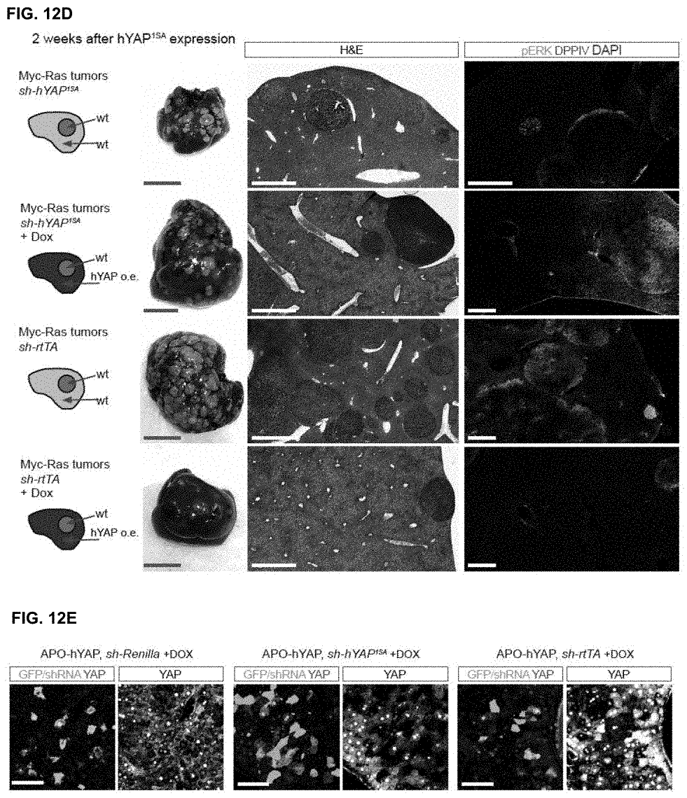

(FIG. 1A) Immunofluorescent detection of YAP on mouse wild-type (top panel) and ICC (intrahepatic cholangiocarcinoma) liver (lower panel) tissue sections. Tumor cells were detected by HA-Akt expression (in green). Arrows and arrow heads indicate Yap in bile duct and portal artery, respectively. Scale bars, 100 μm. (FIG. 1B) Schematic experimental outline. Livers of Yap^{fl/fl};Taz^{fl/fl} mice were hydrodynamically injected with N^{ICD}, HA-Akt and SB11 vectors. At 4 weeks these mice received treatments of tamoxifen (5 consecutive days) and/or AAV-Cre, and were sacrificed and analysed at 7 weeks. (FIG. 1C) Genetic liver schematics (left), whole liver pictures (scale bar, 1 cm) and correspondent haematoxylin-eosin (H&E) (scale bar, 1000 μm) and immunofluorescent stained (right) sections of mouse liver with ICC with conditional Yap and Taz deletion in different liver compartments/tissues. Tumor cells were detected by immunofluorescent staining by HA-Akt expression (in green) (scale bars, 500 μm). Arrows indicate tumors. (FIG. 1D) Quantification of liver to body weight ratios of wild-type, no cre, SB-Cre^{ERT2} and SB-Cre^{ERT2}+AAVCre treated mouse livers at 7 weeks of tumor development. (FIG. 1E) Quantification of the relative percentage of tumor area and (FIG. 1F) absolute tumor load of wild-type and Yap^{-/-};Taz^{-/-} livers with ICC at 7 weeks of tumor development. (FIG. 1G) Western blots for total YAP and TAZ (top) and HA-Akt expression (bottom) of tumor and hepatocyte lysates of wild-type and Yap^{-/-};Taz^{-/-} mice. (FIG. 1H) Immunofluorescent detection of YAP on tumor Yap^{-/-};Taz^{-/-} (top panel) and tumor and hepatocyte Yap^{-/-};Taz^{-/-} (lower panel) tissue sections. Tumor cells were detected by HA-Akt expression (in green). Arrowheads indicate YAP positive endothelial cells. Scale bars, 100 μm. (FIG. 1I) Immunofluorescent detection of tdTomato reporter in wild-type (top), tumor specific recombination (middle) and tumor and hepatocyte recombination (bottom). Scale bars, 100 μm. Data are mean±SEM. See Example 2.1.

FIGS. 2A-2H. YAP is upregulated in peritumoral hepatocytes in mouse and human livers.

(FIG. 2A) Immunofluorescent detection of YAP (left) and TAZ (right) protein expression pattern in livers sections with ICC. Tumor cells were marked by HA-Akt expression (in red). Scale bars, 100 μm. (FIG. 2B) Immunohistochemical detection of YAP on human liver sections. Scale bars, 200 μm. (FIG. 2C) Quantitative RT-PCR for Yap and Taz of normal and peritumoral purified hepatocytes. (FIGS. 2D-2E) Immunofluorescent detection and quantification of liver sections showing changes in ectopic YAP protein localization in wild-type livers and livers with ICC hydrodynamically transfected with HA-tagged YAP t TEAD4. Scale bars, 100 μm. (FIG. 2F) Heatmap showing upregulation of YAP signature genes (Sohn et al. 2016) in peritumoral hepatocytes relative to matched control livers with normal hepatocytes. (FIG. 2G) GSEA plots showing the distribution of two sets of YAP signature genes identified from human HCC (hepatocellular carcinoma) samples (Sohn et al. 2015) and cultured cells overexpressing YAP (Zhao et al. 2008). (FIG. 2H) Validation of YAP signature through quantitative RT-PCR of YAP target genes. See Example 2.2.

FIGS. 3A-3K. YAP in peritumoral hepatocytes acts non-cell autonomously to restrain tumor growth.

(FIGS. 3A and 3B) Immunofluorescent detection and quantification of tdTomato reporter, showing recombination of the R26-tdTomato reporter specifically in hepatocytes (red) but not in tumor cells (marked by HA-Akt, green). Scale bars, 100 μm. (FIG. 3C) Schematic experimental outline. Livers of Yap^{fl/fl};Taz^{fl/fl} mice were hydrodynamically injected with N^{ICD}, HA-Akt and SB11 vectors. At 4 weeks these mice were injected with AAV-Cre, and sacrificed and analysed at 7 weeks. (FIG. 3D) Quantitative RT-PCR for Yap and Taz of wild-type and Yap^{-/-};Taz^{-/-} mice treated with AAV-Cre with and without N-Akt tumors. (FIG. 3E) Western blot of whole liver lysates of wild-type and Yap^{-/-};Taz^{-/-} livers showing efficient deletion of YAP and TAZ. (FIG. 3F) Liver schematics showing genetic manipulations (left), whole liver pictures (scale bar, 1 cm) and correspondent haematoxylin-eosin (H&E) (scale bar, 1000 μm) and immunofluorescent stained (right) sections of mouse liver with ICC with conditional Yap and Taz deletion in hepatocytes. Hepatocytes are marked by HNF [symbol] expression (red) and nuclei by DAPI (grey) (scale bar, 500 μm). (FIG. 3G) Quantification of the average liver to body weight ratios of wild-type and Yap^{-/-};Taz^{-/-} livers with ICC at 7 weeks of tumor development. (FIG. 3H) Quantification of the percentage of relative tumor area of wild-type and Yap^{-/-};Taz^{-/-} livers with ICC at 7 weeks of tumor development. (FIG. 3I) Quantification of the tumor load of wild-type and Yap^{-/-};Taz^{-/-} livers with ICC at 7 weeks of tumor development. (FIGS. 3J-3K) Immunofluorescent analysis and quantification of sections of wild-type and Yap^{-/-};Taz^{-/-} livers showing increased tumor cell proliferation by Ki67 staining (in red) in Yap^{-/-};Taz^{-/-} livers. Tumor cells are marked by HA-Akt expression (in green). Scale bar, 100 μm. Data are mean±SEM. See Example 2.3.

FIGS. 4A-4J. Hyperactivation of YAP in peritumoral hepatocytes induces tumor cell elimination.

(FIG. 4A) Schematic experimental outline. Livers of Lats1^{fl/fl};Lats2^{fl/fl} or Lats1^{fl/fl};Lats2^{fl/fl}; Yap^{fl/fl};Taz^{fl/fl} mice hydrodynamically injected with N^{ICD}, HA-Akt and SB11 vectors. At 4 weeks these mice were injected with AAV-Cre and sacrificed and analysed at 6 weeks. (FIG. 4B) Whole liver and haematoxylin-eosin (H&E) section of a Lats1^{fl/fl};Lats2^{fl/fl} liver at 4 weeks of tumor development. Scale bars, 1 cm and 1000 μm, respectively. (FIG. 4C) Western blot of whole liver lysates showing decreased YAP phosphorylation (5112) and increased TAZ levels in Lats1−/−;Lats2−/− mutant livers compared to wild-type livers in different days after AAV-Cre injection. (FIGS. 4D-4E) Immunofluorescent analysis and quantification of hepatocyte proliferation in sections of wild-type, Lats1−/−;Lats2−/− and Lats1−/−;Lats2−/−; Yap^{-/-};Taz^{-/-} mutant livers. Proliferating cells were marked by Ki67 (red) and tumor cells by HA-tagged Akt (green). Scale bars, 100 μm. (FIG. 4F) Increased liver to body weight ratio of Lats1−/−;Lats2−/− mutant livers in comparison with wild type livers, and rescue in Lats1−/−;Lats2−/−; Yap^{-/-};Taz^{-/-} livers. (FIG. 4G) Liver schematics showing genetic manipulations (left), whole liver pictures (scale bar, 1 cm) and correspondent haematoxylin-eosin (H&E) (scale bar, 1000 μm) and immunofluorescent stained (right) sections of mouse liver with ICC with conditional Lats1/Lats2 and Yap/Taz deletion in hepatocytes. Tumor cells were detected by HA-Akt expression (green) and nuclei by DAPI (blue) (scale bar, 500 μm) (FIG. 4H) Quantification of the relative percentage of tumor area and (FIG. 4I) absolute tumor load of wild-type, Lats1−/−;Lats2−/− and Lats1−/−;Lats2−/−; Yap^{-/-};Taz^{-/-} mutant livers with ICC at 6 weeks of tumor development. (FIG. 4J) Tumor load evolution shows reduction of tumor load in Lats1−/−;Lats2−/− mutant livers after week 5. Data are mean±SEM. See Example 2.4.

FIGS. 5A-5G. YAPISA expression in peritumoral hepatocytes reduces tumor load and extends survival.

(FIG. 5A) Schematic experimental outline. Livers of ApoE-rtTA;TetO-Yap^{1SA} mice were hydrodynamically injected with N^{ICD}, HA-Akt and SB11 vectors. At 4 weeks doxycycline was administered ad libitum for 2 weeks, when mice were sacrificed and analysed. (FIG. 5B) Immunofluorescent detection of Apo>hYAP^{1SA} liver sections, showing hepatocyte-specific human YAP^{1SA} expression (in red) but not in tumor cells (marked by HA-Akt, green). Scale bars, 100 μm. (FIG. 5C) Genetic liver schematics (left), whole liver pictures (scale bar, 1 cm) and correspondent haematoxylin-eosin (H&E) (scale bar, 1000 μm) and immunofluorescent stained (right) sections of mouse liver with ICC with conditional human Yap^{1SA} expression. Tumor cells were detected in immunofluorescent staining by HA-Akt expression (in green) (scale bars, 500 μm). (FIG. 5D) Quantification of the average liver to body weight ratios of wild-type and Apo>YAP'^{1SA}±doxycycline livers with ICC at 6 weeks of tumor development. (FIG. 5E) Quantification of the absolute tumor load of wild-type and Apo>hYAP^{1SA}±doxycycline livers with ICC at 6 weeks of tumor development. (FIG. 5F) Tumor load evolution shows reduction of tumor load in Apo>YAP^{1SA} livers after week S. (FIG. 5G) Percentage of survival of wild-type and Apo>YAP^{1SA} mice with ICC (**, p=0.0033). Data are mean±SEM. See Example 2.5.

FIGS. 6A-6J. BCL2 overexpression in cancer cells prevents tumor elimination induced by Yap activation in peritumoral hepatocytes.

(FIGS. 6A-6B) TUNEL staining (green) and quantification of sections of wild-type and Lats1−/−;Lats2−/− mutant livers with N-Akt tumors, 6 days after AAV-Cre administration. Tumor cells are marked by HA-Akt (red). Scale bars, 100 μm. (FIG. 6C) Quantification of number of cancer associated immune cells (CD45+ and CD3+) in wild-type and Lats1−/−;Lats2−/− liver sections. (FIG. 6D) Western blot of tumors and whole liver lysates for changes in the levels of YAP, TAZ and cleaved Caspase 3. Liver injury caused by CCl⁴ was used as a control sample to detect changes cell death markers. (FIG. 6E) Western blot analysis of tumors from wild-type and Lats1−/−;Lats2−/− mutant livers showing markers of necroptosis, apoptosis and hypoxia. (FIG. 6F) Schematic experimental outline to conditionally induce expression of Bcl2 in tumor cells and delete Lats1 and Lats2. Livers of Lats1^{fl/fl};Lats2^{fl/fl} mice were hydrodynamically injected with N^{ICD}, HA-Akt, TetON-Bcl2 and SB11 vectors. At 4 weeks these mice were injected with AAV-Cre and doxycycline was administered ad libitum for 2 weeks, when mice were sacrificed and analysed. (FIG. 6G) Whole liver pictures (left) (scale bars, 1 cm) and correspondent immunofluorescent stained sections (right) of wild-type and Lats1−/−;Lats2−/− mutant livers bearing ICC tumors with and without expression of Bcl2. Tumor cells were detected by HA-Akt expression (black) and nuclei by DAPI (blue) (scale bars, 500 μm) (FIG. 6H) Average liver to body weight ratios of wild-type and Lats1−/−;Lats2−/− mutant livers bearing ICC tumors with and without expression of Bcl2. (FIG. 6I) Quantification of the relative tumor area in wild-type and Lats1−/−;Lats2−/− mutant livers bearing ICC tumors with and without expression of Bcl2. (FIG. 6J) Quantification of the absolute tumor load in wild-type and Lats1−/−;Lats2−/− mutant livers bearing ICC tumors with and without expression of Bcl2. Data are mean±SEM. See Example 2.6.

FIGS. 7A-7J. YAP activation in peritumoral hepatocytes induces hepatocellular carcinoma and metastatic melanoma cancer cell elimination.

(FIG. 7A) Schematic experimental outline. Livers of ApoE-rtTA;TetO-Yap^{1SA} mice were hydrodynamically injected with Myc-1-NRAS, sh-rtTA, sh-hYAP^{1SA} and SB11 vectors. At 4 weeks doxycycline was administered ad libitum for 2 weeks, when mice were sacrificed and analysed. (FIG. 7B) Quantification of the average liver to body weight ratios of wild-type and Apo>YAP^{1SA}±doxycycline livers with HCC, sh-rtTA and sh-hYAP^{1SA} at 6 weeks. (FIGS. 7C-7D) Quantification of the relative (FIG. 7C) and absolute (FIG. 7D) tumor load of wild-type and Apo>YAP^{15A}±doxycycline livers with HCC, sh-rtTA and sh-hYAP^{1SA} at 6 weeks of tumor development. (FIG. 7E) Liver schematics showing genetic manipulations (left), whole liver pictures (scale bar, 1 cm) and correspondent haematoxylin-eosin (H&E) (scale bar, 1000 μm) and immunofluorescent stained (right) sections of mouse liver with HCC, sh-rtTA and sh-hYAP^{1SA} at 6 weeks with conditional human Yap^{1SA} hepatocyte expression. Tumor cells were detected in immunofluorescent staining positively for phospho-ERK expression (in green) and negatively for DPP-IV (in red) (scale bars, 500 μm) (FIG. 7F) Schematic experimental outline. Livers of ApoE-rtTA;TetO-Yap^{1SA} mice were hydrodynamically injected with 10.000 NRas⁺/INK4a^{-/-} melanoma cells. At 4 weeks doxycycline was administered ad libitum for 2 weeks, when mice were sacrificed and analysed. (FIG. 7G) Quantification of the average liver to body weight ratios of wild-type and Apo>YAP^{A}±doxycycline livers with NRas⁺/INK4a^{-/-} melanoma metastases at 6 weeks. (FIGS. 7H-7I) Quantification of the relative (FIG. 7H) and absolute (FIG. 7I) tumor load of wild-type and Apo>YAP^{1SA}±doxycycline livers with NRas⁺/INK4a^{-/-} melanoma metastases at 6 weeks of tumor development. (FIG. 7J) Genetic liver schematics (left), whole liver pictures (scale bar, 1 cm) and correspondent haematoxylin-eosin (H&E) (scale bar, 1000 μm) and immunofluorescent stained (right) sections of mouse liver with NRas⁺/INK4a^{-/-} melanoma metastases, with conditional human Yap^{1SA} hepatocyte expression. Tumor cells were detected in immunofluorescent staining positively for S100 expression (in green) and negatively for DPPIV (in red) (scale bars, 500 μm). Data are mean±SEM. See Example 2.7.

FIGS. 8A-8F. Tumor cell survival depends on YAP/TAZ levels in surrounding liver tissue.

(FIG. 8A) Whole liver pictures (scale bar, 1 cm) and correspondent haematoxylin-eosin (H&E) (scale bar, 1000 μm) of mouse livers with ICC at different time points (4 to 7 weeks) in wild-type mice. (FIG. 8B) Immunofluorescent stained sections of mouse liver with ICC with Cre in different liver compartments. Tumor cells were detected in immunofluorescent staining by HA-Akt expression (in green) (scale bars, 500 μm). (FIG. 8C) Quantification of the average liver to body weight ratios (FIG. 8D) Quantification of the relative percentage of tumor area and (FIG. 8E) absolute tumor load of wild-type mice with the following treatments: no Cre, SB-Cre^{ERT2} and SB-Cre^{ERT2}+ AAVCre treated mouse livers at 7 weeks of tumor development. (FIG. 8F) Immunofluorescent detection of tdTomato reporter in wild-type (top) and hepatocyte recombination (bottom), showing complete hepatocyte recombination. Scale bars, 100 μm. Data are mean±SEM. See Example 2.1.

FIGS. 9A-9I. YAP is upregulated in peritumoral hepatocytes in mouse and human livers.

(FIG. 9A) Immunohistochemical detection of YAP on human liver sections. Scale bars, 200 μm. (FIGS. 9B-9C) Tables of human HCC and ICC cohorts showing the distribution of patients according to the levels of peritumoral YAP and their etiology. (FIG. 9D) Percentage of patients showing different levels of YAP in peritumoral hepatocytes of human HCC and ICC livers. (FIG. 9E) Schematic experimental outline. Livers of Rosa26 tdTomato mice were hydrodynamically injected with N^{ICD}, HA-Akt and SB11 vectors. At 4 weeks these mice were injected with AAV-Cre and sacrificed and analysed at 7 weeks. (FIG. 9F) FACS dot plot showing purity of Percoll isolated hepatocytes. (FIG. 9G) YAP related gene ontology (GO) terms highly enriched in peritumoral hepatocytes with p-values. (FIGS. 9H-9I) Immunofluorescent analysis and quantification of proliferating hepatocytes between normal livers and liver with ICC. Scale bars, 100 μm. Data are mean±SEM. See Example 2.2.

FIGS. 10A-10D. YAPISA expression in peritumoral hepatocytes reduces tumor load and extends survival.

(FIG. 10A) Increase of average liver to body weight ratios in Apo>hYAP^{1SA}±doxycycline livers over 2 weeks. (FIG. 10B) Immunofluorescent analysis of tumor cell proliferation in sections of Apo>hYAP^{1SA}±doxycycline livers. Proliferating cells were marked by Ki67 (green) and nuclei by DAPI (blue). Scale bars, 100 μm. (FIG. 10C) Western blot of whole liver lysates of wild-type and Apo>hYAP^{1SA}±doxycycline livers showing efficient YAP overexpression. (FIG. 10D) Whole liver picture and haematoxylin-eosin (H&E) sections of wild-type C57BL/6 mouse livers with ICC at 7 weeks. Scale bars, 1 cm and 1000μm, respectively. Data are mean±SEM. See Example 2.5.

FIGS. 11A-11F. BCL2 overexpression in cancer cells prevents tumor elimination induced by Yap activation in peritumoral hepatocytes.

(FIGS. 11A-11B) Immunofluorescent analysis immune cell infiltration in sections of wild-type (top) and Lat1−/−;Lats2−/− (bottom) mutant livers. Immune cells were marked by CD45 and CD3 (red) and tumor cells by HA-tagged Akt (green). Scale bars, 100 μm. (FIG. 11C) Schematic experimental outline to conditionally induce expression of Bcl2 in tumor cells and overexpress hYAP^{1SA}. Livers of Apo>hYAP^{1SA} mice were hydrodynamically injected with N^{ICD}, HA-Akt, TetON-Bcl2 and SB11 vectors. At 4 weeks these mice were injected with AAV-Cre and doxycycline was administered ad libitum for 2 weeks, when mice were sacrificed and analysed. (FIG. 11D) Quantification of the relative tumor area in Apo>hYAP^{1SA}±doxycycline livers bearing ICC tumors with and without expression of Bcl2. (FIG. 11E) Quantification of the absolute tumor load in Apo>hYAP^{1SA}±doxycycline livers bearing ICC tumors with and without expression of Bcl2. (FIG. 11F) Whole liver pictures (left) (scale bars, 1 cm) and correspondent immunofluorescent stained sections (right) of Apo>hYAP's±doxycycline livers bearing ICC tumors with and without expression of Bcl2. Tumor cells were detected by mIgG expression (red) and nuclei by DAPI (blue) (scale bars, 500 μm). Data are mean±SEM. See Example 2.6.

FIGS. 12A-12E. YAP activation in peritumoral hepatocytes induces hepatocellular carcinoma cell elimination.

(FIG. 12A) Schematic experimental outline. Livers of ApoE-rtTA;TetO-Yap^{1SA} mice were hydrodynamically injected with Myc-I-NRAS, sh-Renilla, sh-rtTA or sh-hYAP^{ISA} and SB11 vectors. At 4 weeks doxycycline was administered ad libitum for 2 weeks, when mice were sacrificed and analysed. (FIG. 12B) Quantification of the average liver to body weight ratios of wild-type and Apo>YAP's doxycycline livers with HCC, sh-Renilla, sh-rtTA or sh-hYAP^{1SA} at 6 weeks. (FIG. 12C) Quantification of the absolute tumor load of wild-type and Apo>YAP^{1SA}±doxycycline livers with HCC, sh-Renilla, sh-rtTA or sh-hYAP^{1SA} at 6 weeks of tumor development. (FIG. 12D) Liver schematics showing genetic manipulations (left), whole liver pictures (scale bar, 1 cm) and correspondent haematoxylin-eosin (H&E) (scale bar, 1000 μm) and immunofluorescent stained (right) sections of mouse liver with HCC, sh-Renilla, sh-rtTA or sh-hYAP^{1SA} at 6 weeks with conditional human Yap^{1SA} hepatocyte expression. Tumor cells were detected in immunofluorescent staining positively for phospho-ERK expression (in green) and negatively for DPPIV (in red) (scale bars, 500 μm) (FIG. 12E) Immunofluorescent validation of sh-Renilla, sh-rtTA or sh-hYAP^{1SA} in mouse livers. shRNA expression was detected in immunofluorescent staining positive for GFP (green). Scale bars, 100 μm. Data are mean±SEM. See Example 2.7.

### DETAILED DESCRIPTION TO THE INVENTION

Inhibition of the Hippo pathway effectors YAP and TAZ is emerging as attractive therapeutic intervention. In work leading to the current invention, however, an unexpected tumor suppressive activity of YAP and TAZ themselves was identified. In contrast to hepatocytes in normal livers, peritumoral hepatocytes exhibited high levels of YAP/TAZ activity. Deletion of Yap and Taz in peritumoral hepatocytes caused accelerated growth of liver tumors. Conversely, however, hyperactivation of YAP in peritumoral hepatocytes surprisingly triggered regression of established hepatocellular carcinoma, cholangiocarcinoma, and melanoma-derived metastatic lesions to the liver. The YAP-mediated tumor cell elimination (i.e. tumor elimination mediated by enhanced or activated expression and/or function of YAP and/or TAZ) occurred within days following YAP activation. Thus, a novel mechanism of tumor suppression is identified whereby peritumoral YAP/TAZ activation (i.e. enhanced or activated expression and/or function of YAP and/or TAZ) inhibits tumorigenesis and development of metastases. These findings offer new therapeutic avenues for the treatment in particular of YAP and/or TAZ overexpressing (liver) cancer and (liver) metastasis: activation of a (liver) repair/regeneration response such as caused by hyperactivation of YAP and TAZ in the peritumoral zone (e.g. peritumoral hepatocytes) causes elimination of tumor cells and prevents metastasis. Based hereon, the invention is defined in the following aspects and embodiments, which are described in more detail thereafter.

In one aspect, the invention is relating to enhancers or activators of expression and/or function of YAP and/or TAZ for use in (a method of) treating or inhibiting cancer, for use in (a method of) inhibiting progression of tumor growth, or for use in (a method of) treating or inhibiting tumor metastasis; or for use in the manufacture of a medicament for treating or inhibiting cancer, for inhibiting progression of tumor growth or for treating or inhibiting tumor metastasis. Herein, the enhancer or activator can be activating expression and/or function of YAP and/or TAZ directly or indirectly. In one embodiment, said cancer is liver cancer or said tumor is a liver tumor.

Alternatively, but not necessarily mutually exclusive, the invention is relating to activators of liver regeneration for use in (a method of) treating or inhibiting liver cancer, for use in (a method of) inhibiting progression of liver tumor growth, or for use in (a method of) treating or inhibiting liver tumor metastasis; or for use in the manufacture of a medicament for treating or inhibiting cancer, for inhibiting progression of tumor growth or for treating or inhibiting tumor metastasis. Herein, the activator of liver regeneration may be activating directly or indirectly expression and/or function of YAP and/or TAZ.

In any of the above, the activator of expression and/or function of YAP and/or TAZ or the activator of liver regeneration may be a pharmacologic compound or a gene therapeutic compound as will be explained hereinafter.

When the enhancer or activator of expression and/or function of YAP and/or TAZ or the activator of liver regeneration is a gene therapeutic compound, it can for instance be a nucleic acid capable of enhancing or activating expression and/or function of YAP and/or TAZ, or be a nucleic acid capable of blocking inactivation of expression and/or function of YAP and/or TAZ. Herein, the enhancement or activation of expression and/or function of YAP and/or TAZ can be transient or inducible, or the blocking of inactivation of expression and/or function of YAP and/or TAZ can be transient or inducible. In particular, the gene therapeutic compound can be a nucleic acid capable of driving expression of YAP or of a constitutively active YAP variant; a nucleic acid capable of driving expression of TAZ or of a constitutively active TAZ variant; a nucleic acid capable of driving expression of any combination of YAP, TAZ, constitutively active YAP variant, or constitutively active TAZ variant; or any combination of nucleic acids each individually capable of driving expression of YAP, TAZ, constitutively active YAP variant, or constitutively active TAZ variant. This can optionally be further combined (on a same or separate nucleic acid) with a gene capable of driving expression of a TEAD transcription factor. Requirements for driving expression of a gene include operable linkage between a promoter (such as an organ-specific promoter), the protein-coding sequence, and a terminator.

When the enhancer or activator of expression and/or function of YAP and/or TAZ is a pharmacologic compound, it can for instance be a glucocorticoid, sphingosine-1-phosphate (S1P), dihydro-SiP, lysophosphatidic acid (LPA), or ethacridine. When the activator of liver regeneration is a pharmacologic compound, it can for instance be tri-iodothyronine or a bile acid.

Administration of any of the enhancers or activators of expression and/or function of YAP and/or TAZ or of the activators of liver regeneration for uses (in methods) described hereinabove is to a mammalian subject having a cancer or tumor, and an effective amount of the enhancer or activator or an effective amount of a (pharmaceutically acceptable) composition comprising the enhancer or activator is administered to the mammalian subject in need thereof. In particular, administration of any of the enhancers or activators of expression and/or function of YAP and/or TAZ and/or any of the activators of liver regeneration for uses (in methods) described hereinabove to the mammalian subject can be locally to an organ (such as a liver) having a cancer or tumor, or can be peritumoral, peripheral, or systemic. In case of peripheral or systemic administration, the enhancer or activator can be designed such that it displays tropism to the organ having the cancer or tumor (e.g. by linking in any way to a targeting agent which is targeting the activator to the organ or to cell in the organ).

Any of the enhancers or activators of expression and/or function of YAP and/or TAZ or of the activators of liver regeneration for uses (in methods) described hereinabove, can be administered to a mammalian subject in conjunction or combination (in any type of treatment regimen) with administration of macrophage colony-stimulating factor 1 (CSF1), beta-catenin, granulocyte colony-stimulating factor (GCSF), a RAGE-inhibitor, or in conjunction or combination (in any type of treatment regimen) with administration of any combination thereof, i.e. of any combination of CSF1, beta-catenin, GCSF and/or a RAGE-inhibitor.

Any of the enhancers or activators of expression and/or function of YAP and/or TAZ or of the activators of liver regeneration for uses (in methods) described hereinabove, can be administered to a mammalian subject in conjunction or combination (in any type of treatment regimen) with administration of an attenuator of cell division, an antifibrotic agent, or in conjunction or combination (in any type of treatment regimen) with administration of any combination thereof, i.e. of any combination of an attenuator of cell division and/or an antifibrotic agent. Such combination is in particularly envisaged in case of longer term/more chronic administration of the enhancer or activator in order to reduce side effects such as organ damage or organ disorganization possibly occurring due to prolonged activation of peritumoral cell division.

Any of the enhancers or activators of expression and/or function of YAP and/or TAZ or of the activators of liver regeneration for uses (in methods) described hereinabove, can be administered to a mammalian subject in conjunction or combination (in any type of treatment regimen) in any way with a further anticancer treatment or antitumor agent.

In one particular application, any of the enhancers or activators of expression and/or function of YAP and/or TAZ or of the activators of liver regeneration for uses (in methods) described hereinabove, can be administered to a mammalian subject having a tumor or having cancer prior to surgical intervention or surgical removal of the tumor or cancer. In the latter setting, administration of any of the said enhancers or activators is causing the tumor or cancer to shrink, to regress or to decrease in size or volume, which facilitates subsequent surgical intervention to remove the remaining tumor or cancer, or facilitates subsequent surgical removal of the remaining tumor or cancer.

In a further particular application, any of the enhancers or activators of expression and/or function of YAP and/or TAZ for uses (in methods) described hereinabove, can be administered to a mammalian subject having a tumor or having cancer prior to administration of an inhibitor of expression and/or function of YAP and/or TAZ. In the latter setting, administration of any of the said enhancers or activators is causing the tumor or cancer to shrink, to regress or to decrease in size or volume, and the remaining tumor or cancer is then attacked by the inhibitor of expression and/or function of YAP and/or TAZ. Upon administering the said inhibitor, the effect of enhancement or activation of expression and/or function of YAP and/or TAZ is curtailed (or stopped or inhibited; antidote effect), thus limiting the effect of the enhancers or activators of expression and/or function of YAP and/or TAZ in time, thus limiting the possible side-effects of said enhancers or activators. As such, the inventive concept of the herein described invention (enhancing or activating expression and/or function of YAP and/or TAZ to regress a tumor or cancer) can be combined with the existing intervention in the Hippo signaling pathway aiming at inhibiting YAP and/or TAZ.

In any of the above the organ in particular is the liver, the liver tumor can be a primary liver tumor (e.g. liver cholangiocarcinoma and/or hepatocellular carcinoma) or can be a metastatic liver tumor (secondary liver tumor originating from a non-liver primary tumor).

In any of the above, the enhancer or activator in particular is designed or administered to a mammal in need thereof in such a way that it is capable of inducing peritumoral (liver) cell proliferation.

The aspects and embodiments of the invention described above are supported by the Examples and by the detailed explanation of the terms used in describing the aspects and embodiments as included hereafter.

### Treatment

“Treatment”/“treating” refers to any rate of reduction, delaying or retardation of the progress of the disease or disorder, or of a single symptom thereof, compared to the progress or expected progress of the disease or disorder, or single symptom thereof, when left untreated. More desirable, the treatment results in no/zero progress of the disease or disorder, or single symptom thereof (i.e. “inhibition” or “inhibition of progression”), or even in any rate of regression of the already developed disease or disorder, or single symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

### Tumor, Cancer, Neoplasm; and Liver Cancer

The terms tumor and cancer are sometimes used interchangeably but can be distinguished from each other. A tumor refers to “a mass” which can be benign (more or less harmless) or malignant (cancerous). A cancer is a threatening type of tumor. A tumor is sometimes referred to as a neoplasm: an abnormal cell growth, usually faster compared to growth of normal cells. Benign tumors or neoplasms are non-malignant/non-cancerous, are usually localized and usually do not spread/metastasize to other locations. Because of their size, they can affect neighboring organs and may therefore need removal and/or treatment. A cancer, malignant tumor or malignant neoplasm is cancerous in nature, can metastasize, and sometimes re-occurs at the site from which it was removed (relapse).

The initial site where a cancer starts to develop gives rise to the primary cancer. When cancer cells break away from the primary cancer (“seed”), they can move (e.g. via blood and/or lymph fluid) to another site even remote from the initial site. If the other site allows settlement and growth of these moving cancer cells, a new cancer, called secondary cancer, can emerge (“soil”). The process leading to secondary cancer is also termed metastasis, and secondary cancers are also termed metastases. For instance, liver cancer can arise as primary cancer, but can also be a secondary cancer originating from e.g. a primary breast cancer, bowel cancer or lung cancer; some types of cancer show an organ-specific pattern of metastasis. Most cancer deaths are in fact caused by metastases, rather than by primary tumors (Chambers et al. 2002, Nature Rev Cancer 2:563-572).

In 2012, cancer was the second leading cause of deaths in the USA, but coming very close to the first leading cause being heart diseases. For 2016, the estimated number of new cancer cases (both sexes where relevant) in the USA are, ranked from highest to lowest, breast cancer, lung and bronchus cancer, prostate cancer, colon cancer, skin melanoma and urinary bladder cancer, non-Hodgkin lymphoma, thyroid cancer and kidney and renal pelvis cancer, uterine corpus cancer, pancreas cancer, and rectum cancer and liver and intrahepatic bile duct cancer; jointly about 1.293 million new cases (circa 77% of total expected new cases) (Siegel et al. 2016, CA Cancer J Clin 66:7-30).

Benign liver tumors include hemangioma, hepatic adenoma and focular nodular hyperplasia. Primary liver cancers include hepatocellular carcinoma or hepatoma (HCC) starting from hepatocellular cells; fibrolamellar HCC; cholangiocarcinoma or cholangiocellular carcinoma (CC) or bile duct cancer (intrahepatic or extrahepatic) occurring in bile ducts; angiosarcoma and hemangiocarcinoma starting in liver blood vessels; hepatoblastoma (usually in children); combined hepatocellular and cholangiocarcinomas (cHC-CCs) or other combinations. Secondary liver cancer, or liver metastasis, originating from a primary cancer form an organ or tissue different from the liver. In clinical practice, many secondary liver cancers find their origin from colon or colorectal cancer.

### YAP and TAZ

YAP1, YES-associated protein 1, YAP, YAP2, or YAP65 (used interchangeably) refer to a transcriptional co-factor activating cell proliferation genes and suppressing genes involved in apoptosis. It is part of the Hippo signaling pathway controlling organ size. Different isoforms result from alternative splicing giving rise to multiple transcript variants (Genbank accession numbers given hereafter: isoform 1: NP_001123617.1; isoform 2: NP_006097.2; isoform 3: NP_001181973.1; isoform 4: NP_001181974.1; isoform 5: NP_001269027.1; isoform 6: NP_001269026.1; isoform 7: NP_001269028.1; isoform 8: NP_001269029.1; isoform 9: NP_001269030.1). YAP has been reported to be phosphorylated by the Lats-kinases on Serine residues in a consensus HX(R/H)XXS motif (SEQ ID NO:18); 5 such motifs are present in YAP2, encompassing Ser61, Ser109, Ser127, Ser164 and Ser381 (the relative numbering of these serine residues can vary among YAP-isoforms in a single species, or among YAP proteins of different species). As single mutation, S127A is the strongest constitutively activate YAP variant, but combined mutation of all 5 Serine-residues is most strong constitutively activate YAP variant (Zhao et al. 2007, Genes Dev 21:2747-2761; Iwasa et al. 2013, Exp Cell Res 319:931-945).

TAZ (transcriptional co-activator with PDZ-binding motif; encoding the tafazzin protein) is a close paralog of YAP1 and likewise exists as different isoforms (Genbank accession numbers given hereafter: isoform 1: NP_000107.1; isoform 2: NP_851828.1; isoform 3: NP_851829.1; isoform 4: NP_851830.1). Like YAP, TAZ is comprising consensus motifs HX(R/H)XXS motif (SEQ ID NO:18) (see e.g. FIG. 1 in Hong & Guan 2012, Sem Cell Dev Biol 230:785-793) and constitutively active variants comprising a mutation of the Serine residue in this motif have been reported (e.g. Lei et al 2008, Mol Cell Biol 28:2426-2436).

### Activator/Activation—Enhancer/Enhancement

An enhancer or activator is any compound capable of activating/leading to activation of a process as described herein.

Enhancement or activation of expression of YAP and/or TAZ is referring to an event positively influencing or increasing expression of YAP and/or TAZ at the mRNA and/or protein level.

Enhancement or activation of the function of YAP and/or TAZ is referring to an event negatively influencing or decreasing phosphorylation of YAP- and/or TAZ-protein therewith positively influencing or increasing nuclear localization of YAP- and/or TAZ-protein, or to an event otherwise positively influencing or increasing nuclear localization of YAP- and/or TAZ-protein.

In the above, the enhancement or activation can be direct, meaning that the enhancement or activation of expression of YAP and/or TAZ is through an event at the level of the YAP and/or TAZ gene itself (activation of gene expression or transcription) or at the level of the YAP and/or TAZ mRNA (activation of protein expression or translation); or meaning that the enhancement or activation of the function of YAP and/or TAZ is through an event at the level of the YAP and/or TAZ protein (e.g. protein stabilization, post-translational modification, intracellular trafficking) itself. In case of indirect enhancement or activation, the enhancing or activation signal is an event upstream of the direct enhancing or activation signal, but eventually leads to the direct enhancing or activation signal—indirect enhancement or activation in other words leads through an intermediate event not at the level of the YAP and/or TAZ gene, mRNA or protein itself, but nevertheless resulting in enhancement or activation of the YAP and/or TAZ gene, mRNA or protein.

Enhancement or activation of gene expression is achievable by gene therapeutic means and relies on a genetic construct wherein a suitable promoter, a protein coding sequence and a suitable terminator are included/are operably linked. An organ- or cell-specific promoter can add to the organ- or cell-specific expression of the protein encoded by the operably linked coding sequence.

In the above, the enhancement or activation can be transient, inducible (or alternatively conditional), or can be transient after induction (or alternatively transient after conditional enhancement or activation).

As will be described hereinafter, the enhancement or activation can be triggered by a pharmacologic compound (small molecule, (in)organic molecule, (in)organic compound, peptides and (poly)proteins, modified peptides and (poly)proteins), or by a nucleic acid or nucleic acid comprising compound or alternatively a gene therapeutic compound or alternatively by gene therapy or nucleic acid therapy (used interchangeably); or by a combination of a pharmacologic compound and nucleic acid therapy. A single administration of a pharmacologic compound in general leads to a transient effect due to its gradual removal from the cell, organ and/or body and is reflected in the pharmacokinetic behavior of the compound. Depending on the desired level of activation, two or more (multiple) administrations of the pharmacologic compound may be required. Activation by gene or nucleic acid therapy or by a gene therapeutic (nucleic acid or nucleic acid comprising) compound can be inducible when controlled by a promoter responsive to a to be administered signal not normally present in the target cell, -organ, or -body. As such, the activation by gene or nucleic acid therapy may be transient (e.g. upon removal of the to be administered signal from the target cell, -organ, or -body). In case of a nucleic acid or nucleic acid comprising compound degrading once inside the target cell, -organ, or -body (e.g. in case when not integrated in the genome), the effect of the compound generally is transient.

Enhancement or activation of a process as envisaged in the current invention refers to different possible levels of activation, e.g., at least 10%, at least 20%, at least 30%, at least 40%, at least 50%, at least 60%, at least 70%, at least 80%, at least 90%, at least 95%, or at least 100%, or over 100% of enhancement or activation (compared to a normal situation). The nature of the enhancing or activating compound is not vital/essential to the invention as long as the process envisaged is enhanced or activated such as to treat or inhibit tumor growth or to cause regression of an established tumor.

In case of indirect enhancement or activation as described above, a cellular component or molecule may need to be neutralized, knocked-down or otherwise downregulated (commonly inhibited) in order to achieve the desired enhancement or activation of a process as envisaged in the current invention. Several possible levels of inhibition are envisaged, e.g., at least 10%, at least 20%, at least 30%, at least 40%, at least 50%, at least 60%, at least 70%, at least 80%, at least 90%, at least 95% or even 100% of inhibition (compared to a normal situation). The nature of the inhibitory compound is not vital/essential to the invention as long as the inhibition is such as to treat or inhibit tumor growth or to cause regression of an established tumor.

As described in the Examples section hereinafter, hyperactivation of YAP and TAZ was leading to hepatocyte proliferation. In the Examples section, one way of YAP and TAZ hyperactivation was obtained by downregulation of Lats1- and Lats2-expression. In a patient setting, it is feasible to obtain such downregulation in a conditional and/or transient manner (see further) by e.g. administering to the hepatocytes shRNA or siRNA targeting Lats1 and/or Lats2. Obviously, YAP and/or TAZ themselves(s) can be conditionally and/or transiently overexpressed. Alternatively, variants of YAP and/or TAZ such as the gain-of-function/constitutively active human YAP Ser127Ala mutant (Camargo et al. 2007, Curr Biol 17:2054-2060; Dong et al. 2007, Cell 130:1120-1133; see also above) can be conditionally and/or transiently overexpressed. In principle, any component of the Hippo pathway upstream of YAP and/or TAZ is amenable to trigger indirect activation of YAP and/or TAZ expression and/or function by means of pharmacologic compounds or nucleic acid therapy. As outlined in the Examples herein, the presence of a tumor in the liver caused ectopically (over)expressed YAP to localize in the nucleus (functional activation), and co-expression of TEAD1 (also known as transcriptional enhancer factor TEF-1, TEA domain family member 1, or transcription factor 13 (TCF-13)) likewise caused ectopically (over)expressed YAP to localize in the nucleus (functional activation). Following hereafter is a non-exhaustive list of other ways to obtain higher than normal expression and/or activation of YAP and TAZ, and of other ways to induce liver regeneration.

Pharmacologic compounds such as synthetic glucocorticoids such as betamethasone, hydrocortisone and dexamethasone were demonstrated to by inducers of YAP protein expression (Sorrentino et al. 2016, Nat Comm 8:14073). Other small molecule activators of YAP expression and/or YAP function include sphingosine-1-phosphate (S1P), dihydro-SiP, and lysophosphatidic acid (LPA) (Miller et al. 2012, Chem Biol 19:955-962).

In promoting dephosphorylation of TAZ, the organic molecule ethacridine was identified as an activator of TAZ. Ethacridine is a widely used antiseptic and abortifacient agent (Kawano et al. 2015, J Biochem 158:413-423).

If needed, overexpression of YAP can be countered by administration of YAP-inhibitors. This can happen to counter, mitigate, reduce or prevent side-effects of prolonged activation of YAP and/orTAZ expression or function. It can also be part of a therapeutic strategy (see above) comprising initial activation of YAP and/or TAZ expression and/or function to shrink tumor size, followed by inhibiting or reducing YAP and/or TAZ expression and/or function, the latter at the same time countering, mitigating, reducing or preventing side-effects of prolonged activation of YAP and/or TAZ expression or function.

Inhibitors of YAP and/or TAZ include molecules such as antiparasitic macrocyclic lactones (e.g. ivermectin, milbemycin D) (Nishio et al. 2016, Proc Natl Acad Sci USA 113:E71-E80), porphyrin- and dipyrrin-related derivatives (e.g. verteporfin) (Gibault et al. 2017, Chem Med Chem 12:954-961), or statins (e.g. simvastatin) (Wang et al. 2014, Proc Natl Acad Sci USA 111:E89-E98). Different types of small molecule or peptidic YAP/TAZ inhibitors are discussed in Calces et al. 2019 (Trends in Cancer 5:297-307). YAP and/or TAZ inhibitors include verteporfin (Liu-Chittenden et al. 2012, Genes Dev 26:1300-1305), a small molecule referred to as CA3 (Song et al. 2018, Mol Cancer Ther 17,443-454), and fluorene-oxime compounds disclosed in WO2017058716 and WO2018204532. Other YAP and/or TAZ inhibitors interfere with the binding of YAP and/or TAZ with the TEAD transcription factors YAP and/or TAZ control (TEAD1-4). Patents on small molecule inhibitors of the YAP/TAZ-TEAD interaction have been summarized by Crawford et al. 2018, Expert Opin Ther Pat 28:867-873. Such inhibitors include compounds based on a bis-aryl hydrazine scaffold as disclosed in WO2017064277 and WO2018185266. Alternatively, such inhibitors target a lipid pocket at the core of all four TEADs, which is generally occupied by a palmitoyl ligand and is essential for TEAD folding, stability, and YAP binding; see e.g. WO2017/053706. Flufenamic acid or derivatives thereof, such as derivatives comprising chloromethyl ketone moieties and binding to the conserved cysteine in the lipid pocket of TEADs have also been reported as inhibitors of the interaction of YAP and/or TAZ with TEADs (Pobbati et al. 2015, Structure 23:2076-2086; Bum-Erdene et al. 2018, Cell Chem Biol 26:378-389). Further reported as YAP-TAZ/TEAD inhibitors are cyclic YAP-like peptides (Zhang et al. 2014, ACS Med Chem Lett 5, 993-998) and peptides mimicking VGLL4 (Vestigial-like protein 4) (Jiao et al. 2014, Cancer Cell 25:166-180). Dasatinib, statins, and pazopanib have been reported to inhibit the nuclear localization and target gene expression of YAP and TAZ, and are thus a further type of YAP and/or TAZ inhibitors (Oku et al. 2015, FEBS Open Bio 5:542-549). Downregulating overexpressed YAP- and/or TAZ-expression is likewise feasible through gene therapy (e.g., by administering siRNA, shRNA or antisense oligonucleotides to YAP and/or TAZ), or by administering a pharmacological inhibitor of YAP and/or TAZ (e.g. an antibody). This will be described in more detail further herein. Upregulation of expression of inhibitors of YAP and/or TAZ expression and/or function is also feasible through gene therapy, e.g. by upregulation of Lats1- and/or Lats2-expression, or by downregulation of expression of TEAD factors (see Background section).

### Liver Regeneration

In a healthy situation, liver cells are relatively quiescent. Activation of liver regeneration therefore is referring to an event positively influencing or increasing liver cell proliferation. Several factors can trigger or contribute to liver cell proliferation (reviewed by Tao et al. 2017, Mediators Inflammation, Article ID 4256352).

Inducing liver regeneration is a clinically relevant process (Forbes & Newsome 2016, Nature Genetics 13:473-485) and efforts gone into its understanding are applicable in the context of the current invention. Pharmacologically, liver regeneration can be induced by e.g. administration of tri-iodothyronine (T3). Forbes et al 1998 (Gene Ther 5:552-555) applied this in the context of rendering hepatocytes receptive for retroviral-based integrative gene transfer (requiring replicating cells). T3 administration was later reported to have the advantage of suppressing neocarcinogenesis, a possible side effect of prolonged induction of liver regeneration (Perra et al 2009, Hepatology 49:1287-1296). It has also been hypothesized that too rapid liver regeneration may lead to structurally disorganized tissue. Controlling the rate of liver regeneration, in particular slowing this rate down, was proposed as a means of avoiding such unwanted effects. An ERK1/2 inhibitor and a selective MEK inhibitor were applied to such effect (Ninomiya et al. 2010, Am J Transplant 10:1580-1587). Fibrosis potentially associated with rapid liver regeneration could be suppressed by administering losartan (Colmenero et al. 2009, Am J Physiol Gastrointest Liver Physiol 297:G726-G734).

Elevation of bile acid levels have also been reported to stimulate liver regeneration. In mice, feeding for 5 days with 0.2% cholic acid leads, without toxic effects, to a liver size increase of 30%. Bile acids are thought to be (one of) the soluble circulating signal(s) responsible for causing hepatocyte proliferation in a healthy animal parabiotically linked to an animal subjected to partial hepatectomy (Huang et al. 2006, Science 312: 233-236). Bile acids are sensed by the nuclear bile acid receptor FXR (farnesoid X receptor), and overexpression of FXR or STAT3 protects from subsequent liver injury (Meng et al. 2010, Mol Endocrinol 24:886-897).

Sufficient levels of macrophage colony stimulating factor (M-CSF/CSF1), and thus of M-CSF-induced Kupffer cells (liver tissue macrophages) need to be present to support liver regeneration. These Kuppfer cells appear to be the source of IL-6 required for priming liver regeneration (Amemiya et al. 2011, J Surg Res 165:59-67; Tao et al. 2017, Mediators Inflammation: 4256352). In case of acute liver failure, the serum level of CSF1 is a prognostic marker for patient survival, possibly linked to immune function of liver macrophages (Stutchfield et al. 2015, Gastroenterology 149:1896-1909).

Similarly, β-catenin levels need to be sufficiently high to support liver regeneration, at least in inured livers (Apte et al. 2009, Am J Pathol 175:1056-1065).

Granulocyte colony stimulating factor (G-CSF) has also been reported to contribute to liver regeneration, at least in injured livers, and possibly without causing fibrosis (Yannaki et al. 2005, Exp Hematol 33:108-119; Garg et al. 2012, Gastroenterology 142:505-512).

Finally, blocking RAGE (receptor for advanced glycation end product), e.g. by administering soluble RAGE (sRAGE), may also contribute to efficient liver regeneration (Zeng et al. 2004, Hepatology 39:422-432).

Unwanted effects of activation of liver cell proliferation can be counteracted by e.g. administering a compound slowing down the proliferation rate (such as for instance a cell cycle inhibitor such as a small molecule, a peptide or a nucleic acid; e.g. Dickson & Schwartz 2009, Current Oncol 16:36-43; Peyressatre et al. 2015, Cancers 7:179-237; Jin et al. 1995, Cancer Res 55:3250-3253; or such as inhibitors of CDK4-6 (cell cycle dependent kinase) such as PD 0332991 (Flaherty et al. 2012, Clin Cancer Res 18:568-576) or ribociclib or palbociclib) or administering an antifibrotic compound (see e.g. Rosenbloom et al. 2013, Biochem Biophys Acta 1832:1088-1103). In general the activation of liver cell proliferation is limited in time. The time should be sufficient to allow the intended therapeutic effect (treatment or inhibition of liver cancer or inhibition of progression of liver cancer, wherein the liver cancer is primary liver cancer or liver cancer starting out of metastases from other cancers) to occur, after which the activated proliferation should gradually fade out/be switched off/be inactivated to return to normal, i.e. to return the proliferating liver cells to their quiescent or near quiescent state. The latter is important to prevent potential side effects of continued liver cell proliferation (too large liver, increased chance of development of liver cancer).

### Liver Targeting

Local drug administration in the liver or in the vicinity of a liver tumor (peritumoral) is feasible such as by using e.g. a minimally invasive catheter permitting repetitive administration. Furthermore, hydrodynamic delivery favors hepatocytic uptake. Endowing a pharmacologic compound or gene therapeutic compound with liver cell-targeting properties may enable peripheral and/or systemic administration of such compound.

Extensive work has already been performed on liver cell-targeted delivery of compounds and of liver cell-specific gene therapy. This has been summarized by e.g. Kang et al. 2016 (Crit Rev Biotechnol 36:132-143) and Poelstra et al. 2012 (J Controlled Rel 161: 188-197). Much of this work is related to treatment of liver fibrosis, liver cirrhosis, hepatitis and HCC. Problems raised concern potential relative poor selectivity of the liver targeting strategies towards diseased cells. In the framework of the current invention, such problems are expected to be less of a concern as in fact the healthy liver cells are targeted.

Without reproducing the whole of Kang et al. 2016 (Crit Rev Biotechnol 36:132-143) or Poelstra et al. 2012 (J Controlled Rel 161: 188-197), strategies have been designed for targeting hepatocytes, Kupffer cells, sinusoidal endothelial cells and stellate cells (e.g. Table 1 of Kang et al. 2016). By targeting the mannose/N-acetylglucosamine receptor for instance, compounds can be delivered to hepatocytes, Kupffer cells and sinusoidal endothelial cells. By means of using a (recombinant) hepatitis B virus pre-S1 protein or pre-S1-derived peptide for instance, or by means of (recombinant) hepatitis B virus L protein nanocapsules (possibly with one or more substitutions of cysteine for another amino acid)(Nagaoka et al. 2007, J Control Rel 118:348-356), compounds can be targeted to hepatocytes.

For instance, co-injection of N-acetylglucosamine-conjugated melittin-like peptide together with a cholesterol-conjugated siRNA lead to efficient knockdown of the targeted gene (Wooddell et al. 2013, Mol Ther 21:973-985). Antisense oligonucleotides (formulated in Lipofectin) have also been applied to reduce gene expression in liver cells (e.g. Zhang et al. 2000, Nat Biotechnol 18:862-867; Zhang et al. 2003, J Pharmacol Exp Ther 307:24-33). Passive targeting was used in the case of pegylated interferon alpha-2b (PEG-intron) wherein the pegylation causes prolonged circulation leading to prolonged uptake by liver cells (reviewed in Poelstra et al. 2012, J Controlled Rel 161: 188-197). A long list of nanocarriers, lipoplexes, liposomes, or polyplexes designed to deliver genes specifically to liver cells (and often including cationic polymers such as polyethylenimine, polyallylamine, poly-L-lysine or chitosan) is provided in e.g. Table 1 of Pathak et al. 2008 (Int J Nanomed 3:31-49).

Non-viral gene delivery in liver cells may be hampered by degradation within liver cell lysosomes. Including a fusogenic peptide (as lysosome disruptive element) in a non-viral gene delivery agent was reported to increased efficiency of this administration modality. On the other hand, nanoparticles (such as of PLGA) were reported to escape lysosomes (reviewed in Pathak et al. 2008, Int J Nanomed 3:31-49).

Adenovirus-associated virus (AAV; in particular gutless AAV), such as AAV2 and AAV8, has been shown to be suitable for shuttling genetic information or gene therapeutic compounds into liver cells. The stronger tropism of AAV8 (moreover having a lower seroprevalence in humans compared to AAV2) for the liver allows peripheral administration of a gene therapeutic compound (as used in the Examples herein). Higher gene expression was observed using a gene expression cassette packaged in AAV as complementary dimers (self-complementary) compared to single-stranded AAV expression cassettes (Nathwani et al. 2011, N Engl J Med 365:2357-2365). AAV is a non-integrative vector and therefore disappears together with turnover of the transfected cells. Whereas normal liver cells are quiescent, the current invention envisages in one aspect enhancing their replication (regeneration response), thus leading to dilution of the transgene delivered through AAV-transfection, thus diminishing its overall effect and contributing to transient transgene expression.

Liver-specificity of expression of gene therapeutic compounds can also, or in addition, be obtained by using liver-specific gene promoters. The liver-specific AAT-apolipoprotein E (apoE) promoter (hAAT promoter and four copies of the human apo E enhancer, or derivatives thereof; hMAT=human α₁ antitrypsin) is one such example (Van Linthout et al. 2002, Hum Gene Ther 13:829-840). Other reported liver-specific promoters are one comprising two copies of alpha 1 microglobulin/bikunin enhancer coupled to the core promoter of human thyroxine-binding globulin (TBG), and one comprising randomly assembled hepatocyte-specific transcription factor binding sites linked to the murine transthyretin promoter (reviewed in Kattenhorn et al. 2016, Hum Gene Ther 27:947-961).

Regulation of expression of a transgene delivered to liver cells is feasible, e.g., by means of using tet-dependent expression (as also used in the Examples as described hereinafter). This was e.g. applied in a lentiviral vector (such vectors can integrate stably in both dividing and non-dividing cells) administered to liver cells (Vigna et al. 2005, Mol Ther 11:763-775).

Besides AAV and lentiviruses, retroviruses (e.g. HIV), hemagglutinating virus of Japan (HVJ), and hepatitis B viral particles have been applied in liver gene therapy. Modifications include e.g. HIV vectors pseudotyped with Sendai virus fusion protein F and fusion of HVJ with cationic liposomes to arrive at virosomes (reviewed in Poelstra et al. 2012, J Controlled Rel 161: 188-197). Adenoviral vectors, AAV vectors, lentiviral vectors and murine leukemia retroviral vectors for use in in vivo and in ex vivo transfer to liver cells have been reviewed by Nguyen and Ferry 2004 (Gene Ther 11:576-584), as well as hepatocyte transplantation (possibly after ex vivo gene transfer).

Liver targeting can be applied to the activators of the processes envisaged herein, in particular to activators of liver regeneration and to activators of expression and/or function of YAP- and/or TAZ. The AAV8 vector, and the apoE- and TBG-promoters were used in the Examples as outlined hereafter.

### Nucleic Acid or Gene Therapy

Interest in nucleic acid-based therapies has increased over the years. Key in (viral) DNA-based therapy is the presence in the vector of transcription signals enabling production of translatable mRNA in the target cell. In view of concerns regarding the safety of DNA and vector-based therapy, the use of antigen-encoding translatable (m)RNA for vaccination has gained traction. Compared to viral vectors or plasmid DNA, (m)RNA-based therapy present several advantages. In lacking the ability to integrate in the host genome, it is presumed to be much safer (no inadvertent mutations, and transient expression of the encoded protein leading to controlled antigen exposure and minimization tolerance induction). Potentially foreign sequences such as plasmid backbone or viral promotors are not required, reducing the risk in raising an immune response. Further, it offers the possibility to transfect slow or non-dividing cells as RNA does not need to cross the nuclear barrier for protein expression. Adaptation to result in transient, or in the alternative, inducible expression, or in a further alternative inducible transient expression of the target protein and/or targeted delivery of the nucleic acid to the tumor, cancer or neoplasm all are envisaged herein. Direct intracellular or intra-organ delivery represents a further method of targeted delivery.

Methods for administering nucleic acids include methods applying non-viral (DNA or RNA) or viral nucleic acids (DNA or RNA viral vectors). Methods for non-viral gene therapy include the injection of naked DNA (circular or linear), electroporation, the gene gun, sonoporation, magnetofection, the use of oligonucleotides, lipoplexes (e.g. complexes of nucleic acid with DOTAP or DOPE or combinations thereof, complexes with other cationic lipids), dendrimers, viral-like particles, inorganic nanoparticles, hydrodynamic delivery, photochemical internalization (Berg et al. 2010, Methods Mol Biol 635:133-145) or combinations thereof.

Many different vectors have been used in human nucleic acid therapy trials and a listing can be found on httn://www.abedia.com/wiley/vectors.php. Currently the major groups are adenovirus or adeno-associated virus vectors (in about 21% and 7% of the clinical trials, respectively), retrovirus vectors (about 19% of clinical trials), naked or plasmid DNA (about 17% of clinical trials), and lentivirus vectors (about 6% of clinical trials). Combinations are also possible, e.g. naked or plasmid DNA combined with adenovirus, or RNA combined with naked or plasmid DNA to list just a few. Other viruses (e.g. alphaviruses) are used in nucleic acid therapy and are not excluded in the context of the current invention.

Administration may be aided by specific formulation of the nucleic acid e.g. in liposomes (lipoplexes) or polymersomes (synthetic variants of liposomes), as polyplexes (nucleic acid complexed with polymers), carried on dendrimers, in inorganic (nano)particles (e.g. containing iron oxide in case of magnetofection), or combined with a cell penetrating peptide (CPP) to increase cellular uptake. Organ- or cellular-targeting strategies may also be applied to the nucleic acid (nucleic acid combined with organ- or cell-targeting moiety); these include passive targeting (mostly achieved by adapted formulation) or active targeting (e.g. by coupling a nucleic acid-comprising nanoparticle with any compound (e.g. an aptamer, antibody or fragment thereof, antigen binding molecule, monobody, affitin, anticalin, DARPin, alphabody, single domain antibody or fragment thereof) binding to a target organ- or cell-specific antigen) (e.g. Steichen et al. 2013, Eur J Pharm Sci 48:416-427).

CPPs enable translocation of the drug of interest coupled to them across the plasma membrane. CPPs are alternatively termed Protein Transduction Domains (PTDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody (or other agent; see above) binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

Any other modification of the DNA or RNA to enhance efficacy of nucleic acid therapy is likewise envisaged to be useful in the context of the applications of the nucleic acid or nucleic acid comprising compound as outlined herein. The enhanced efficacy can reside in enhanced expression, enhanced delivery properties, enhanced stability and the like. The applications of the nucleic acid or nucleic acid comprising compound as outlined herein may thus rely on using a modified nucleic acid as described above, or as described in the next section.

### Hypoinflammatory Nucleic Acids

A known problem with e.g. adenoviral nucleic acid therapy is its triggering of an inflammatory response. Less inflammatory (hypoinflammatory) helper-dependent or gutless adenovirus vectors, can alternatively be used as hypoinflammatory adenoviral vector for nucleic acid therapy. Other solutions include covalent modification of the viral capsid proteins (e.g. by PEGylation), modifying the adenoviral fiber knob (composition), vector encapsulation in a polymer, and/or serotype switching or reverting to non-human adenoviral vectors (e.g. Ahi et al. 2011, Curr Gene Ther 11:307-320).

Naked DNA nucleic acid therapy can likewise provoke inflammatory responses. Linear DNA from which the bacterial backbone sequences were removed was reported to be less inflammatory (hypoinflammatory) than linear DNA comprising the bacterial backbone sequences and to be less inflammatory than circular DNA (Zhu et al. 2009, Biomed Pharmacother 63:129-135). Reducing the amount of unmethylated CpG motifs or sequential injection of cationic liposomes followed by naked plasmid DNA are other alternatives to arrive at hypoinflammatory DNA therapy (Niidome & Huang 2002, Gene Therapy 9:1647-1652).

In case of RNA-based expression constructs, it was also reported that they can induce inflammatory immune responses which could ameliorate their efficacy. Kariko et al. 2005 (Immunity 23:165-175) established that modified to heavily modified eukaryotic RNA is not immunostimulatory compared to nearly unmodified RNA (eukaryotic or other). On the other hand, mRNA lacking poly(A)-tail is also immunostimulatory (even from a eukaryotic source). This led to the suggestion of including naturally occurring modified nucleosides (more than 100 exist, a list is available on http://mods.rna.albany.edu/mods/), such as 5-methylcytidine and pseudouridine, in therapeutic RNA (Pollard et al. 2013 Mol Ther 21:251-259). Hypoinflammatory RNA as referred to herein is heterologous RNA constructed such as to minimize potential inflammatory responses by including naturally occurring modified nucleosides wherein the modified nucleosides are preferably unique to and frequently used in RNA of the species in which the heterologous hypoinflammatory RNA is to be administered.

### Modulation of Expression or Function

One process of modulating expression of a gene of interest relies on antisense oligonucleotides (ASOs), or variants thereof such as gapmers. An antisense oligonucleotide (ASO) is a short strand of nucleotides and/or nucleotide analogues that hybridizes with the complementary mRNA in a sequence-specific manner via Watson-Crick base pairing. Formation of the ASO-mRNA complex ultimately results in downregulation of target protein expression. Depending on the target sequence, ASOs can act in different ways. If the ASO is taken up by cellular endocytosis and hybridizes with target mRNA in the cytoplasm, formation of an ASO-mRNA complex can induce activation of RNase H (selective degradation of bound mRNA) or can sterically interference with ribosomal assembly. In case the ASO can enter the nucleus, mRNA maturation can be modulated by inhibition of 5′ cap formation, inhibition of mRNA splicing or activation of RNaseH (Chan et al. 2006, Clin Exp Pharmacol Physiol 33:533-540; this reference also describes some of the software available for assisting in design of ASOs). Modifications to ASOs can be introduced at one or more levels: phosphate linkage modification (e.g. introduction of one or more of phosphodiester, phosphoramidate or phosphorothioate bonds), sugar modification (e.g. introduction of one or more of LNA (locked nucleic acids), 2′-O-methyl, 2′-O-methoxy-ethyl, 2′-fluoro, S-constrained ethyl or tricyclo-DNA and/or non-ribose modifications (e.g. introduction of one or more of phosphorodiamidate morpholinos or peptide nucleic acids). The introduction of 2′-modifications has been shown to enhance safety and pharmacologic properties of antisense oligonucleotides. Antisense strategies relying on degradation of mRNA by RNase H requires the presence of nucleotides with a free 2′-oxygen, i.e. not all nucleotides in the antisense molecule should be 2′-modified. The gapmer strategy has been developed to this end. A gapmer antisense oligonucleotide consists of a central DNA region (usually a minimum of 7 or 8 nucleotides) with (usually 2 or 3) 2′-modified nucleosides flanking both ends of the central DNA region. This is sufficient for the protection against exonucleases while allowing RNAseH to act on the (2′-modification free) gap region. Antidote strategies are available as demonstrated by administration of an oligonucleotide fully complementary to the antisense oligonucleotide (Crosby et al. 2015, Nucleic Acid Ther 25:297-305).

Another process to modulate expression of a gene of interest is based on the natural process of RNA interference. It relies on double-stranded RNA (dsRNA) that is cut by an enzyme called Dicer, resulting in double stranded small interfering RNA (siRNA) molecules which are 20-25 nucleotides long. siRNA then binds to the cellular RNA-Induced Silencing Complex (RISC) separating the two strands into the passenger and guide strand. While the passenger strand is degraded, RISC is cleaving mRNA specifically at a site instructed by the guide strand. Destruction of the mRNA prevents production of the protein of interest and the gene is ‘silenced’. siRNAs are dsRNAs with 2 nt 3′ end overhangs whereas shRNAs are dsRNAs that contains a loop structure that is processed to siRNA. shRNAs are introduced into the nuclei of target cells using a vector (e.g. bacterial or viral) that optionally can stably integrate into the genome. Apart from checking for lack of cross-reactivity with non-target genes, manufacturers of RNAi products provide guidelines for designing siRNA/shRNA. siRNA sequences between 19-29 nt are generally the most effective. Sequences longer than 30 nt can result in nonspecific silencing. Ideal sites to target include AA dinucleotides and the 19 nt 3′ of them in the target mRNA sequence. Typically, siRNAs with 3′ dUdU or dTdT dinucleotide overhangs are more effective. Other dinucleotide overhangs could maintain activity but GG overhangs should be avoided. Also to be avoided are siRNA designs with a 4-6 poly(T) tract (acting as a termination signal for RNA pol III), and the G/C content is advised to be between 35-55%. shRNAs should comprise sense and antisense sequences (advised to each be 19-21 nt in length) separated by loop structure, and a 3′ AAAA overhang. Effective loop structures are suggested to be 3-9 nt in length. It is suggested to follow the sense-loop-antisense order in designing the shRNA cassette and to avoid 5′ overhangs in the shRNA construct. shRNAs are usually transcribed from vectors, e.g. driven by the Pol III U6 promoter or H1 promoter. Vectors allow for inducible shRNA expression, e.g. relying on the Tet-on and Tet-off inducible systems commercially available, or on a modified U6 promoter that is induced by the insect hormone ecdysone. A Cre-Lox recombination system has been used to achieve controlled expression in mice. Synthetic shRNAs can be chemically modified to affect their activity and stability. Plasmid DNA or dsRNA can be delivered to a cell by means of transfection (lipid transfection, cationic polymer-based nanoparticles, lipid or cell-penetrating peptide conjugation) or electroporation. Viral vectors include lentiviral, retroviral, adenoviral and adeno-associated viral vectors.

Ribozymes (ribonucleic acid enzymes) are another type of molecules that can be used to modulate expression of a target gene. They are RNA molecules capable of catalyzing specific biochemical reactions, in the current context capable of targeted cleavage of nucleotide sequences. Examples of ribozymes include the hammerhead ribozyme, the Varkud Satellite ribozyme, Leadzyme and the hairpin ribozyme. Besides the use of the inhibitory RNA technology, modulation of expression of a gene of interest can be achieved at DNA level such as by gene therapy to knock-out or disrupt the target gene. As used herein, a “gene knock-out” can be a gene knockdown or the gene can be knocked out by a mutation such as, a point mutation, an insertion, a deletion, a frameshift, or a missense mutation by techniques such as described hereafter, including, but not limited to, retroviral gene transfer. Another way in which genes can be knocked out is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of the endogenous DNA repair machinery, these reagents can be used to precisely alter the genomes of higher organisms.

Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). Another recent genome editing technology is the CRISPR/Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway. Recently, it was demonstrated that the CRISPR-Cas editing system can also be used to target RNA. It has been shown that the Class 2 type VI-A CRISPR-Cas effector C2c2 can be programmed to cleave single stranded RNA targets carrying complementary protospacers (Abudayyeh et al. 2016 Science 353/science.aaf5573). C2c2 is a single-effector endoRNase mediating ssRNA cleavage once it has been guided by a single crRNA guide toward the target RNA.

Interfering with structure, which can result in inhibition or activation of function, can be achieved by e.g. binding moieties binding to the protein of interest. Non-limiting examples are (monoclonal) antibodies or antigen-binding fragments thereof, alpha-bodies, nanobodies, intrabodies (antibodies binding and/or acting to intracellular target; this typically requires the expression of the antibody within the target cell, which can be accomplished by gene therapy), aptamers, DARPins, affibodies, affitins, anticalins, monobodies, phosphatases (in case of phosphorylated target) and kinases (in case of a phosphorylatable target).

The term “antibody” as used herein refers to any naturally occurring format of antibody or antigen binding protein the production of which is induced by an immune system (immunoglobulins or IgGs). It is clear, however, that not all antibodies are naturally occurring as e.g. some antigens are problematic in the sense that they are poor or not at all immunogenic, or are not recognized by the immune system (e.g. self-antigens); artificial tricks may be required to obtain antibodies against such antigens (e.g. knock-out mice: e.g. Declercq et al. 1995, J Biol Chem 270:8397-8400; DNA immunization for e.g. transmembrane antigens; e.g. Liu et al. 2016, Emerg Microbes Infect 5:e33). “Conventional” antibodies comprise two heavy chains linked together by disulfide bonds and two light chains, one light chain being linked to each of the heavy chains by disulfide bonds. Each heavy chain has at one end a variable domain (VH) followed by a number of constant domains (three or four constant domains, CH1, CH2, CH3 and CH4, depending on the antibody class). Each light chain has a variable domain (VL) at one end and a constant domain (CL) at its other end; the constant domains of the light chains each align with the first constant domains of the heavy chains, and the light chain variable domains each align with the variable domains of the heavy chains. This type of antibodies exist in camels, dromedaries and llamas along with an “unconventional” naturally occurring type of antibodies consisting of only two heavy chains, and thus being devoid of light chains. Other “unconventional” naturally occurring antibodies exist in in the serum of nurse sharks (Ginglymostomatidae) and wobbegong sharks (Orectolobidae). These latter antibodies are called Ig new antigen receptors (IgNARs). They are disulfide-bonded homodimers consisting of five constant domains (CNAR) and one variable domain (VNAR). There is no light chain, and the individual variable domains are independent in solution and do not appear to associate across a hydrophobic interface (Greenberg et al. 1995, Nature 374:168-173; Nuttall et al. 2001, Mol Immunol 38:313-326; Diaz et al. 2002, Immunogenetics 54:501-512; Nuttall et al. 2003, EurJ Biochem 270:3543-3554). Due to the heavy chain dimer structure characteristic of camelid and shark antibodies, these are sometimes termed “Heavy-Chain Mini-Antibodies” (mnHCAbs) or simply “Mini-Antibodies” (mnAbs) (Holliger & Hudson 2005, Nature Biotechnol 23:1126-1136). The complementary determining region 3 (CDR3) of camel antibodies and shark antibodies is usually longer (comprising about 16-21 amino acids, and about 16-27 amino acids, respectively) than the CDR3 of mouse VH region (comprising about 9 amino acids) (Muyldermans et al. 1994, Prot Eng 7:1129-1135; Dooley & Flajnik 2005, Eur J Immunol 35:936-945). Without the light chain, these heavy-chain antibodies bind to their antigens by one single domain, the variable antigen binding domain of the heavy-chain immunoglobulin, referred to as Vab (camelid antibodies) or V-NAR (shark antibodies). These smallest intact and independently functional antigen binding fragment Vab is referred to as nano-antibody or nanobody (Muyldermans 2001, J Biotechnol 74:277-302). Multivalent (etc. divalent, trivalent, tetravalent and pentavalent) Vab and/or V-NAR domains may be preferred in some instances due to their potentially higher cellular intake and retention and may be made by recombinant technology or by chemical means, such as described in WO 2010/033913. The variable domains of the light and/or heavy chains are involved directly in binding the antibody to the antigen. The variable domains of naturally occurring light and heavy chains have the same general structure: four framework regions (FRs) connected by three complementarity determining regions (CDRs) (see e.g. Kabat et al. 1991, Sequences of Proteins of Immunological Interest, 5 thEd. Public Health Service, National Institutes of Health, Bethesda, Md.). The CDRs in a light or heavy chain are held in close proximity by the FRs and contribute to the formation of the antigen binding site. An antibody, or antibody fragment as described hereafter, may also be part of a multivalent and/or multispecific antigen binding molecule. An overview of e.g. available bispecific formats (around 100) is provided in Brinkmann & Kontermann 2017 (mAbs 9:182-212). The term “antibody fragment” refers to any molecule comprising one or more fragments (usually one or more CDRs) of an antibody (the parent antibody) such that it binds to the same antigen to which the parent antibody binds. Antibody fragments include Fv, Fab, Fab′, Fab′-SH, single-chain antibody molecules (such as scFv), F(ab′) 2, single variable VH domains, and single variable VL domains (Holliger & Hudson 2005, Nature Biotechnol 23:1126-1136), Vab and V-NAR. The term further includes microantibodies, i.e. the minimum recognition unit of a parent antibody usually comprising just one CDR (Heap et al. 2005, J Gen Virol 86:1791-1800). Any of the fragments can be incorporated in a multivalent and/or multispecific larger molecule, e.g. mono- or bi-specific Fab 2, mono- or tri-specific Fab 3, bis-scFv (mono- or bispecific), diabodies (mono- or bi-specific), triabodies (e.g. trivalent monospecific), tetrabodies (e.g. tetravalent monospecific), minibodies and the like (Holliger & Hudson 2005, Nature Biotechnol 23:1126-1136). Any of the fragments can further be incorporated in e.g. V-NAR domains of shark antibodies or VhH domains of camelid antibodies (nanobodies). All these are included in the term “antibody fragment”.

Alphabodies are also known as Cell-Penetrating Alphabodies and are small 10 kDa proteins engineered to bind to a variety of antigens.

Aptamers have been selected against small molecules, toxins, peptides, proteins, viruses, bacteria, and even against whole cells. DNA/RNA/XNA aptamers are single stranded and typically around 15-60 nucleotides in length although longer sequences of 220 nt have been selected; they can contain non-natural nucleotides (XNA) as described for antisense RNA. A nucleotide aptamer binding to the vascular endothelial growth factor (VEGF) was approved by FDA for treatment of macular degeneration. Variants of RNA aptamers are spiegelmers are composed entirely of an unnatural L-ribonucleic acid backbone. A Spiegelmer of the same sequence has the same binding properties of the corresponding RNA aptamer, except it binds to the mirror image of its target molecule. Peptide aptamers consist of one (or more) short variable peptide domains, attached at both ends to a protein scaffold, e.g. the Affimer scaffold based on the cystatin protein fold. A further variation is described in e.g. WO 2004/077062 wherein e.g. 2 peptide loops are attached to an organic scaffold. Phage-display screening of such peptides has proven to be possible in e.g. WO 2009/098450.

DARPins stands for designed ankyrin repeat proteins. DARPin libraries with randomized potential target interaction residues, with diversities of over 10 variants, have been generated at the DNA level. From these, DARPins can be selected for binding to a target of choice with picomolar affinity and specificity.

Affitins, or nanofitins, are artificial proteins structurally derived from the DNA binding protein Sac7d, found in Sulfolobus acidocaldarius. By randomizing the amino acids on the binding surface of Sac7d and 5 subjecting the resulting protein library to rounds of ribosome display, the affinity can be directed towards various targets, such as peptides, proteins, viruses, and bacteria.

Anticalins are derived from human lipocalins which are a family of naturally binding proteins and mutation of amino acids at the binding site allows for changing the affinity and selectivity towards a 10 target of interest. They have better tissue penetration than antibodies and are stable at temperatures up to 70° C.

Monobodies are synthetic binding proteins that are constructed starting from the fibronectin type III domain (FN3) as a molecular scaffold.

Based on the above, molecules selected from the group consisting of an antisense oligonucleotide, a gapmer, a siRNA, a shRNA, an antisense oligonucleotide, a zinc-finger nuclease, a meganuclease, a TAL effector nuclease, a CRISPR-Cas effector, an antibody or a fragment thereof (binding to the same antigen as the full-length antibody), an alpha-body, a nanobody, an intrabody, an aptamer, a DARPin, an affibody, an affitin, an anticalin, and a monobody, can be applied in order to activate a process as envisaged in the context of the present invention, more in particular in the activation of liver regeneration or in the activation of expression and/or function of YAP and/or TAZ.

In the above, the molecules are specific to their intended target, which is referring to the fact that the molecules are acting at the level of the intended target and not at the level of target different from the intended target. Specificity can be ascertained by e.g. determining physical interaction of the molecules to their intended target.

### Combination Therapy

The therapeutic modality of the current invention (be it a pharmacologic compound, nucleic acid, or nucleic acid comprising compound) can be combined (simultaneously or in any order; in any treatment regimen) with one or more other antitumor, anticancer or antineoplastic therapy in a combination therapy. Several types of antitumor, anticancer or antineoplastic therapy are listed hereunder. It will be clear, however, that none of these lists is meant to be exhaustive and is included merely for illustrative purposes.

As referred to hereinabove, administration of a therapeutic modality of the current invention could for instance occur at the time of surgical removal of the tumor, cancer or neoplasm (debulking the tumor, cancer or neoplasm mass) although it may be preferred to perform the administration of the therapeutic modality of the current invention prior to surgical removal in order to provide sufficient time and/or sufficient (remaining) tumor, cancer or neoplasm cells for the therapeutic potential of the therapeutic modality of the current invention to develop. In many, if not all, cases a biopsy is taken of a tumor, cancer or neoplasm; as this procedure provides access to the tumor, cancer or neoplasm, the therapeutic modality of the current invention could be administered at this timepoint. Combination of administration of the therapeutic modality of the current invention with radiation therapy or chemotherapy can also be envisaged.

Without being exhaustive, antitumor, anticancer or antineoplastic agents include alkylating agents (nitrogen mustards: melphalan, cyclophosphamide, ifosfamide; nitrosoureas; alkylsulfonates; ethyleneimines; triazene; methyl hydrazines; platinum coordination complexes: cisplatin, carboplatin, oxaliplatin), antimetabolites (folate antagonists: methotrexate; purine antagonists; pyrimidine antagonists: 5-fluorouracil, cytarabibe), natural plant products (Vinca alkaloids: vincristine, vinblastine; taxanes: paclitaxel, docetaxel; epipodophyllotoxins: etoposide; camptothecins: irinotecan), natural microorganism products (antibiotics: doxorubicin, bleomycin; enzymes: L-asparaginase), hormones and antagonists (corticosteroids: prednisone, dexamethasone; estrogens: ethinyloestradiol; antiestrogens: tamoxifen; progesteron derivative: megestrol acetate; androgen: testosterone propionate; antiandrogen: flutamide, bicalutamide; aromatase inhibitor: letrozole, anastrazole; 5-alpha reductase inhibitor: finasteride; GnRH analogue: leuprolide, buserelin; growth hormone, glucagon and insulin inhibitor: octreotide). Other antineoplastic or antitumor agents include hydroxyurea, imatinib mesylate, epirubicin, bortezomib, zoledronic acid, geftinib, leucovorin, pamidronate, and gemcitabine.

Without being exhaustive, antitumor, anticancer or antineoplastic antibodies (antibody therapy) include rituximab, bevacizumab, ibritumomab tiuxetan, tositumomab, brentuximab vedotin, gemtuzumab ozogamicin, alemtuzumab, adecatumumab, labetuzumab, pemtumomab, oregovomab, minretumomab, farletuzumab, etaracizumab, volociximab, cetuximab, panitumumab, nimotuzumab, trastuzumab, pertuzumab, mapatumumab, denosumab, and sibrotuzumab.

A particular class of antitumor, anticancer or antineoplastic agents are designed to stimulate the immune system (immune checkpoint or other immunostimulating therapy). These include so-called immune checkpoint inhibitors or inhibitors of co-inhibitory receptors and include PD-1 (Programmed cell death 1) inhibitors (e.g. pembrolizumab, nivolumab, pidilizumab), PD-Li (Programmed cell death 1 ligand) inhibitors (e.g. atezolizumab, avelumab, durvalumab), CTLA-4 (Cytotoxic T-lymphocyte associated protein 4; CD152) inhibitors (e.g. ipilimumab, tremelimumab) (e.g. Sharon et al. 2014, Chin J Canc 33:434-444). PD-1 and CTLA-4 are members of the immunoglobulin superfamily of co-receptors expressed on T-cells. Inhibition of other co-inhibitory receptors under evaluation as antitumor, anticancer or antineoplastic agents include inhibitors of Lag-3 (lymphocyte activation gene 3), Tim-3 (T cell immunoglobulin 3) and TIGIT (T cell immunoglobulin and ITM domain) (Anderson et al. 2016, Immunity 44:989-1004). Stimulation of members of the TNFR superfamily of co-receptors expressed on T-cells, such as stimulation of 4-1BB (CD137), OX40 (CD134) or GITR (glucocorticoid-induced TNF receptor family-related gene), is also evaluated for antitumor, anticancer or antineoplastic therapy (Peggs et al. 2009, Clin Exp Immunol 157:9-19).

Further antitumor, anticancer or antineoplastic agents include immune-stimulating agents such as—or neo-epitope cancer vaccines (neo-antigen or neo-epitope vaccination; based on the patient's sequencing data to look for tumor-specific mutations, thus leading to a form of personalized immunotherapy; Kaiser 2017, Science 356:112; Sahin et al. 2017, Nature 547:222-226) and some Toll-like receptor (TLR) ligands (Kaczanowska et al. 2013, J Leukoc Biol 93:847-863).

Yet further antitumor, anticancer or antineoplastic agents include oncolytic viruses (oncolytic virus therapy) such as employed in oncolytic virus immunotherapy (Kaufman et al. 2015, Nat Rev Drug Discov 14:642-662), any other cancer vaccine (cancer vaccine administration; Guo et al. 2013, Adv Cancer Res 119:421-475), and any other anticancer nucleic acid therapy (wherein “other” refers to it being different from therapy with a nucleic acid or nucleic acid comprising compound already specifically envisaged in the current invention).

Therefore, in any of the aspects and embodiments of the invention, the therapeutic modality of the current invention may be further combined with another therapy against the tumor, cancer or neoplasm. Such other therapies include for instance surgery, radiation, chemotherapy, immune checkpoint or other immunostimulating therapy, neo-antigen or neo-epitope vaccination, cancer vaccine administration, oncolytic virus therapy, antibody therapy, or any other nucleic acid therapy targeting the tumor, cancer or neoplasm.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

The term “defined by SEQ ID NO:X” as used herein refers to a biological sequence consisting of the sequence of amino acids or nucleotides given in the SEQ ID NO:X. For instance, an antigen defined in/by SEQ ID NO:X consists of the amino acid sequence given in SEQ ID NO:X. A further example is an amino acid sequence comprising SEQ ID NO:X, which refers to an amino acid sequence longer than the amino acid sequence given in SEQ ID NO:X but entirely comprising the amino acid sequence given in SEQ ID NO:X (wherein the amino acid sequence given in SEQ ID NO:X can be located N-terminally or C-terminally in the longer amino acid sequence, or can be embedded in the longer amino acid sequence), or to an amino acid sequence consisting of the amino acid sequence given in SEQ ID NO:X.

The aspects and embodiments described above in general may comprise the administration of an activator to a mammal in need thereof, i.e., harboring a tumor, cancer or neoplasm in need of treatment. In general a (therapeutically) effective amount of an activator is administered to the mammal in need thereof in order to obtain the described clinical response(s). The (therapeutically) effective amount of activator will depend on many factors such as route of administration and tumor mass and will need to be determined on a case-by-case basis by the physician. In general the maximum dose of (therapeutically) effective amount of activator that may be administered to a mammal is determined by the possible toxicity of the activator and is reflected in the maximum tolerated dose (MTD), i.e. the highest dose of activator that does not cause unacceptable side effects. “Administering” means any mode of contacting that results in interaction between an agent (e.g. an activator as described herein) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

The term “effective amount” refers to the dosing regimen of the agent (e.g. activator as described herein) or composition comprising the agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects. To obtain or maintain the effective amount, the agent or composition comprising the agent may be administered as a single dose or in multiple doses. The effective amount may further vary depending on the severity of the condition that needs to be treated; this may depend on the overall health and physical condition of the mammal or patient and usually the treating doctor's or physician's assessment will be required to establish what is the effective amount. The effective amount may further be obtained by a combination of different types of contacting or administration.

The group of mammals includes, besides humans, mammals such as primates, cattle, horses, sheep, goats, pigs, rabbits, mice, rats, guinea pigs, llama's, dromedaries and camels, as well as to mammalian pet animals (dogs, cats, gerbils, hamsters, chinchillas, ferrets etc.).

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

The content of the documents cited herein are incorporated by reference.

### EXAMPLES

### 1. Materials and Methods

### Mouse Strains

Yap^{flox/flox}; Taz^{flox/flox} mouse lines were generated by Erik Olson and gifted by R. L. Johnson (Xin et al. 2013; Xin et al. 2011). Lats1^{flox/flox}; Lats2^{flox/flox} were generated and gifted by R. L. Johnson (Heallen et al. 2013; Heallen et al. 2011). Rosa26-Iox-stop-Iox-tdTomato mice were kindly provided by C. Marine (Madisen et al. 2010). ApoE-rtTA,TRE-Yap mouse line was generated and gifted by D. Pan. L (Dong et al. 2007). Lats1^{flox/flox}; Lats2^{flox/flox}; Yap^{flox/flox}; Taz^{flox/flox} mice were generated in house by crossing the Lats1^{flox/flox}; Lats2^{flox/flox} and Yap^{flox/flox}; Taz^{flox/flox} lines (described above). C57BL/6 mice were purchased from Charles River. Controls matched for sex and age were littermates. Mice were housed, fed and treated in accordance with protocols approved by the committee for animal research at KULeuven. All mouse experiments were approved by the institutional ethical commission at KULeuven and were performed in accordance with relevant institutional and national guidelines and regulations.

### Patient Samples

Liver biopsies were obtained from patients with HCC or ICC at University Hospitals Leuven. All samples were collected after obtaining written informed consent. Immediately after surgical removal of the tumor (resection samples), the tissue was fixed in 6% formalin and embedded in paraffin. The histopathological diagnosis of HCCs or combined ICCs was performed according to the World Health Organization criteria. The study was approved by the ethical committee of the University Hospitals of Leuven, Belgium.

### Plasmids

Plasmids expressing human myc tagged TEAD4 (Myc-TEAD4) and hyperactive sleeping beauty (pCMV/SB11) were obtained from Addgene (#24638 and #26552, respectively). The fragments of mouse myristoylated and HA-tagged AKT, myc-tagged NOTCHI receptor and inducible CreERT2 were obtained from the pT3-EF1α-myr-HA-AKT (Addgene #31789), pT3-EF1α-myc-NICD (Addgene #86500) and pCAG-CreERT2 (Addgene #14797) vectors, respectively, and subcloned into the SfiI restriction sites of a sleeping beauty vector pSBbi-puro (Addgene #60523). The human Bcl2 gene fragment was obtained from the FLAG-Bcl2 vector (Addgene #18003) by PCR and subcloned into SfiI restriction sites of an inducible sleeping beauty vector pSBtet-RH (Addgene #60500). Human YAP fragment was obtained from the pEGFP-C3-YAP2 (Addgene #19055) vector by PCR; an HA-tag was added into the 5′primer, and the PCR product was subcloned into the NotI and XbaI restriction sites of the pcDNA3.1 vector (Invitrogen). The pCaMIN plasmid expressing mouse Myc and NRAS^{G12V} was generated and gifted by L. Zender. shRNAs targeting human YAP cDNA and rTTA^{s}-M2 were ordered as 97 bp ultramers from IDT and cloned into modified pRRL-LT3-GEPIR vector as previously described (Fellmann et al. 2013). For constitutive expression, shRNA-carrying pRRL-LT3-GEPIR vectors were digested by NcoI-NheI and obtained GFP-miR-E fragments were cloned into pSBbi-Puro vector by using NcoI-XbaI sites.

### Generation of Melanoma Mouse Cell Line

Melanoma from Tyr::N-Ras^{Q61K} Ink4a^{-/-} (Tyr::Cre^{ERT2}) animals was dissociated into small pieces using forceps and scissors. Tissue was digested using collagenase I (2 mg/ml, Sigma Aldrich, cat. C0130) and IV (2 mg/ml, Sigma Aldrich, cat. C5138) mix for 20 min at 37° C. followed by a Trypsin (Trypsin-EDTA 0.05%, ThermoFisher Scientific, cat. 25300054) digestion for 5 min at 37° C. Single cells were separated from remaining tissue using a 40 μm cell strainer and cultured in vitro using Dulbecco's Modified Eagle's Medium (DMEM) supplemented with 10% fetal bovine serum and 100 μg/mL Penicillin/Streptomycin.

### Hydrodynamic Tail Vein Injection

For intrahepatic delivery of the transposon system (Kang et al., 2011), eight to ten-week-old mice were secured and hydrodynamically injected with 1 μg pCMV/SB11, 4 μg pSBbi-puro-myrAKT-HA, 20 μg of pSBbi-puro-myc-NICD, or 10 μg of cMyc-IRES-NRas via lateral tail vein. For the expression of SB-Cre^{ERT2} or inducible Bcl2, 15 μg of pSBbi-puro-Cre^{ERT2} or 10 μg of pSBTetON—RH-Flag-Bcl2, respectively, were added to the plasmid mixture. All plasmids were diluted in sterile filtered 0.9% NaCl, and the total volume was adjusted to 10% (in ml) of the total body weight (in grams).

For intrahepatic seeding of tumor cells, mice were hydrodynamically injected with 10.000 mouse melanoma cells (NRas⁺/INK4a^{-/-}) diluted in sterile minimal essential medium (MEM), adjusted to 10% (in ml) of total body weight (in grams). All injected mice were monitored daily and sacrificed in groups at appropriate time points. All experimental and control groups contained 5 to 10 mice.

### Doxycycline and Tamoxifen Administration

For conditional deletion of Yap and Taz, tamoxifen was administered for 5 consecutive days, via intraperitoneal injection at a concentration of 1.6 mg/kg in corn oil. In order to activate the expression of TetON-Bcl2 or human YAP expression in Apo>hYAP^{1SA} mice, 0.2 mg/ml of doxycycline was diluted in filtered sterile drinking water supplemented with 2.5% sucrose and administered ad libido. Doxycycline containing water bottles were protected from light and replaced every other day.

### AAV8-Cre Administration

Adeno-associated virus serotype 8 (AAV8) expressing CRE recombinase under the hepatocyte specific promoter TBG was purchased from UPenn (AAV8.TBG.PI.Cre.rBG, catalog AV-8-PV1091). Mice received 5×10¹¹ GC of AAV8.TBG.PI.Cre.rBG diluted in 200 ul of 1× phosphate-buffered saline (PBS) by tail vein injection.

### Histology and Immunohistochemistry

Human formalin-fixed paraffin-embedded tissue slides (5 μm thick) were stained with antibodies against YAP and TAZ. Visualization was done using DAB-Chromogen, followed by a haematoxylin counterstaining. Mouse livers were fixed with 4% paraformaldehyde (PFA) for 48 hours at 4° C. Paraffin sections (6 μm) were used for histology and were stained with haematoxylin and eosin (H&E) and imaged using Slide Scanner Axio Scan.Z1 microscope.

### Immunofluorescence Staining and Image Analysis

For immunofluorescence analysis, livers samples were embedded in 4% agarose in PBS and then sectioned at 100 μm thickness using a Vibratome (model Leica VT 10005). Liver sections were permeabilized with 0.5% TritonX-100 for 10 minutes and blocked in 3% Bovine Serum Antibody (BSA) in PBS for 2 hours at room temperature. The sections were then incubated in primary antibody solution overnight. The following day, sections were incubated in secondary antibody solution for 2 hours at room temperature. Sections were then washed and mounted in Mowiol and analyzed on an Olympus FV1200 confocal microscope. Images were processed in ImageJ with Bio-Formats Importer plug-in.

### Hepatocyte Isolation

Mice were anesthetized by intraperitoneal injection of sodium pentobarbital (Nembutal, 50 mg/kg). Livers were perfused for 5 minutes with 40 ml of perfusion medium SC-1 to remove the blood, followed by perfusion with 30 ml of SC-2 medium containing 10 mg of collagenase for 5 minutes. Each lobe was dissected off and minced into small pieces in a beaker containing 60 ml of SC-20, 20 mg of collagenase (Roche), and 1 ml of DNase I (Sigma) followed by rotating incubation for 20 min at 37° C. The cells were then filtered through a 70 μm strainer and centrifuged at 500 rpm for 2 min at room temperature. Hepatocytes were re-suspended in 5 ml of SC-2 medium, applied on top of a 25% Percoll solution, and centrifuged for 20 minutes at 4° C.

### Quantitative RT-qPCR

Total RNA was isolated from mouse tissues using an RNeasy Mini Kit (Qiagen) according to manufacturer's instructions. RNA samples were reverse-transcribed to complementary DNA with oligo dT and RevertAid H Minus Reverse Transcriptase (Thermo Scientific). Real-time quantitative PCR was performed using LC 480 SYBR Green I Master (Roche) reaction. All runs were run in triplicate and expression levels were normalized to Gapdh levels. Detailed list of primers is provided in the Key resources table.

### Western Blot

Whole liver samples, micro dissected tumor and hepatocyte samples were sonicated in RPPA buffer supplemented with protease inhibitor cocktail (Sigma). Protein concentration was determined using BCA assay (Pierce). Lysates were diluted in Laemmli sample buffer, 20 μg of protein sample were loaded on SDS-PAGE gels and transferred onto PVDF membranes (Millipore). Protein bands were visualized by using enhanced chemiluminescence film (GE Healthcare Life Sciences). Densitometric quantifications of bands were done using ImageJ software.

RNA-Seq Analysis In total, 6 samples were used in this RNA-seq analysis. The 3 replicates of peritumoral hepatocytes (Peri Tu) are defined as: PH_WT_tumors_r1, PH_WT_tumors_r2, PH_WT_tumors_r3. The following samples were used as controls (3 replicates) for peritumoral hepatocytes (Peri Tu Ctrls): PH_WT_notreat_r1, PH_WT_notreat_r2, PH_WT_notreat_r3. Raw sequencing reads were cleaned for adapters usingfastq-mcf. The cleaned reads were mapped to the Mus Musculus 10 genome (GRCm38/mm10) and assigned to genes using STAR: RNA-seq aligner (Dobin et al. 2013). The raw counts matrix (obtained by STAR) was filtered for low expressed genes (less than one count per sample). Heatmaps were generated on log 2 normalized median centered expression data using Multiple Experiment Viewer (MeV)(Saeed et al. 2003). Principle Component Analysis was performed using the pre-filtered count data transformed to the log 2 scale. Differential gene expression analysis was then performed using the DESeq2 R package version 1.16.1 (Love et al. 2014).

### Gene Set Enrichment

To build the gene rankings for peritumoral hepatocytes, genes were ranked based on the statvalue. The Gene set enrichment was performed using the GSEA software v3.0 (Mootha et al. 2003; Subramanian et al. 2005).

### Data and Software Availability

The accession number for the single cell sequencing datasets reported in this paper is GEO: GSE103788.

### Quantifications and Statistics

All quantifications were performed using ImageJ software. Quantification of relative tumor area was done by measuring the tumor area (marked by HA-Akt positive cells in N-Akt model, phospho-ERK expression in Myc-Ras model or S100 expression in melanoma model) and the total area (marked by DAPI). In addition, tumor luminal spaces were excluded in this measurement since these do not contribute to the tumor cell mass. We inferred absolute tumor load by multiplying the tumor area by the liver weight of each mouse (absolute tumor load (cm³)=tumor area (cm)×liver weight (grams).

Quantifications of cell death and cell proliferation were done using ImageJ Cell Counter plugin. All statistical analyses were performed using Prism7 (Graphpad). The data are presented as the mean±SEM and unpaired Student's t-tests was used for analysis of each time-point, unless stated otherwise. A p value of 0.05 was considered statistically significant. *, **, and *** correspond to p values of <0.05, 0.01 and 0.001, respectively.

### Data and Software Availability

The accession number for the single cell sequencing datasets reported in this application is GEO:

GSE103788.
Oligonucleotides
pSBbi-puro-sh1rtTA
(SEQ ID NO: 1)
TGCTGTTGACAGTGAGCGACAACAGAGAAACAGTACGAAATAGTGAAGCC
ACAGATGTATTTCGTACTGTTTCTCTGTTGGTGCCTACTGCCTCGGA
pSBbi-puro-sh2rtTA
(SEQ ID NO: 2)
TGCTGTTGACAGTGAGCGAGCCCTTGACGATTTTGACTTATAGTGAAGCC
ACAGATGTATAAGTCAAAATCGTCAAGGGCGTGCCTACTGCCTCGGA
pSBbi-puro-sh3rtTA
(SEQ ID NO: 3)
TGCTGTTGACAGTGAGCGCCAGGAGCATCAAGTAGCAAAATAGTGAAGCC
ACAGATGTATTTTGCTACTTGATGCTCCTGTTGCCTACTGCCTCGGA
pSBbi-puro-sh1hYAP
(SEQ ID NO: 4)
TGCTGTTGACAGTGAGCGCCGACAGTCTTCTTTTGAGATATAGTGAAGCC
ACAGATGTATATCTCAAAAGAAGACTGTCGATGCCTACTGCCTCGGA
pSBbi-puro-sh2hYAP
(SEQ ID NO: 5)
TGCTGTTGACAGTGAGCGCACAGGTGATACTATCAACCAATAGTGAAGCC
ACAGATGTATTGGTTGATAGTATCACCTGTATGCCTACTGCCTCGGA
mm Yap1 qPCR forward primer:
(SEQ ID NO: 6)
GCCATGTTGTTGTCTGATCG
mm Yap1 qPCR reverse primer:
(SEQ ID NO: 7)
CCTGATGATGTACCACTGCC
mm TAZ qPCR forward primer:
(SEQ ID NO: 8)
TGCCATGTGGTGATTTTCTC
mm TAZ qPCR reverse primer:
(SEQ ID NO: 9)
CCTATGACGTGACCGACGAG
mm Ctgf qPCR forward primer:
(SEQ ID NO: 10)
GCTTGGCGATTTTAGGTGTC
mm Ctgf qPCR reverse primer:
(SEQ ID NO: 11)
CAGACTGGAGAAGCAGAGCC
mm Cyr61 qPCR forward primer:
(SEQ ID NO: 12)
TTTACAGTTGGGCTGGAAGC
mm Cyr61 qPCR reverse primer:
(SEQ ID NO: 13)
CACCGCTCTGAAAGGGATCT
mm ANKRD1 qPCR forward primer:
(SEQ ID NO: 14)
TGAGGCTGAACCGCTATAAGA
mm ANKRD1 qPCR reverse primer:
(SEQ ID NO: 15)
CAGTGCAACACCAGATCCAT
mm Gapdh qPCR forward primer:
(SEQ ID NO: 16)
CGTCCCGTAGACAAAATGGT
mm Gapdh qPCR reverse primer:
(SEQ ID NO: 17)
TTGATGGCAACAATCTCCAC

| REAGENT OR RESOURCE | SOURCE | IDENTIFIER |
|---|---|---|
| Antibodies | | |
| Actin, α-Smooth Muscle | Sigma | Cat #C6198 |
| Beta-Actin | Abcam | Cat #ab8227 |
| BCL2 | Cell Signaling CST | Cat #3498S |
| BNIP3 | Cell Signaling CST | Cat #3769S |
| Cleaved-Caspase3 (Asp175) | Cell Signaling CST | Cat #9661L |
| CD3 | Abcam | Cat #ab5690 |
| CD45 | Abcam | Cat #ab10558 |
| DPPIV/CD26 | R&D systems | Cat #AF954 |
| GLUT1 | Cell Signaling CST | Cat #12939S |
| HA-Tag | Cell Signaling CST | Cat # 3724S |
| HIF2∝ | Novus biologicals/Bio Connect | Cat #NB100-132 |
| HNF4∝ | Santa Cruz | Cat #sc-6556 |
| Ki67 | Abcam | Cat #ab15580 |
| MLKL | Biorbyt | Cat #32399 |
| Phospho-p44/42 MAPK (Erk1/2) | Cell Signaling CST | Cat # 9101s |
| (Thr202/Tyr204) | | |
| Phospho-MLKL (Ser345) | Cell Signaling CST | Cat #37333S |
| Phospho-RIP3 (Thr231/Ser232) | Cell Signaling CST | Cat # 57220S |
| Phospho-YAP (ser 127) | Cell Signaling CST | Cat #4911S |
| RIP3 | Novus biologicals/Bio Connect | Cat #NBP1-77299 |
| S100 | Dako | Cat #Z0311 |
| TAZ: anti-WWTR1 | Sigma | Cat #HPA007415 |
| tdTomato (16D7) | Kerafast | Cat #EST203 |
| YAP1 | Abcam | #ab52771 |
| Donkey anti-Mouse IgG (H + L) | Thermo Fisher | Cat #A-31571 |
| Bacterial and Virus Strains | | |
| AAV8.TBG.PI.Cre.rBG | Penn Vector Core | AV-8-PV1091 |
| Biological Samples | | |
| Human liver samples | UZ Leuven, Belgium | N/A |
| Chemicals, Peptides, and Recombinant | | |
| Proteins | | |
| Doxycycline hyclate | Sigma | Cat #D9891 |
| Tamoxifen | Sigma | Cat #T5648 |
| Critical Commercial Assays | | |
| DeadEnd ™ Fluorometric TUNEL System | Promega | Cat #G3250 |
| Deposited Data | | |
| Gene Expression Omnibus (GEO) | N/A | GSE103788 |
| Experimental Models: Organisms/Strains | | |
| Gt(ROSA)26Sortm14(CAG-tdTomato)Hze | Provided by C. Marine | N/A |
| | (Madisen et al., 2010) | |
| Yap1<tm1.1Eno>; Wwtr1<tm1.1Eno> | Provided by R. L. Johnson | N/A |
| | (Xin et al., 2013; Xin et al., 2011) | |
| STOCK Lats1tm1.1Jfm/RjoJ; STOCK | Provided by R. L. Johnson | N/A |
| Lats2tm1.1Jfm/RjoJ | | |
| ApoE-rtTA,TRE-Yap | Provided by D. Pan | N/A |
| | (Dong et al., 2007) | |
| Yap1<tm1.1Eno>; Wwtr1<tm1.1Eno>; STOCK | This application | N/A |
| Lats1tm1.1Jfm/RjoJ; STOCK Lats2tm1.1Jfm/RjoJ | | |
| Tyr::N-Ras^{Q61K} Ink4a^{-/-} (Tyr::Cre^{ERT}) | This application | N/A |
| Recombinant DNA | | |
| pCMV/SB11 | Addgene (Perry Hackett) | Cat #26552 |
| myc-TEAD4 | Addgene (Kun Liang Guan) | Cat #24638 |
| pCaMIN | Provided by L. Zender | N/A |
| | (Seehawer et al., 2018) | |
| pSBTet-RH-Flag-Bcl2 | This application | N/A |
| pSBbi-puro-myrAKT-HA | This application | N/A |
| pSBbi-puro-HAYAP | This application | N/A |
| pSBbi-puro-mycNICD | This application | N/A |
| pSBbi-puro-CreERT2 | This application | N/A |
| Software | | |
| ImageJ 1.51m9 | https://imagej.nih.gov/ij/ | N/A |
| Adobe Photoshop CS6 | Adobe Systems | N/A |
| Multi experiment viewer (MEV) 4.8 | http://mev.tm4.org/ | N/A |
| Prism 7 | GraphPad Software, Inc. | N/A |
| | https://www.graphpad.com/scientific-software/prism/ | |

### 2. Results

### 2.1. The Reliance of Liver Cancer Cells on YAP/TAZ for Survival Depends on the Activation of YAP/TAZ in Peritumoral Hepatocytes

To study the function of YAP and TAZ in and around liver tumors, we used mouse models involving the somatic transformation of scattered hepatocytes in adult mice via hydrodynamic tail vein injection of genome-integrating sleeping beauty (SB) plasmids that drive expression of various (activated) oncogenes. We first induced the development of intrahepatic cholangiocarcinoma (ICC) by co-expressing activated versions of the Notch receptor (Notch intracellular domain, N^{ICD}) and Akt (myristolated and HA-tagged, HA-Akt) (Fan et al., 2012). Injection of these plasmids lead to multiple macroscopic tumors 6-7 weeks after DNA injection (FIG. 8A, hereafter referred to as N-Akt tumors) (Fan et al. 2012).

As observed in human cholangiocarcinoma (Kim et al. 2013; Marti et al. 2015; Pei et al. 2015), mouse N-Akt tumor cells had high levels of YAP and TAZ (FIG. 1A). YAP levels in tumor cells were as high as those in bile ducts and endothelial cells where YAP is highly expressed (FIG. 1A)(Wang et al. 2017; Zhang et al. 2010). To test the importance of YAP and TAZ for tumor maintenance, we deleted Yap, and its homolog Taz to eliminate potential compensatory redundancy, in established N-Akt tumors. We co-injected a plasmid that expressed the tamoxifen inducible Cre^{ERT2} (SB-Cre^{ERT2}) together with the N^{ICD} and HA-Akt plasmids into Yap^{fl/fl};Taz^{fl/fl} double floxed mice, and then triggered Yap and Taz deletion by tamoxifen administration four weeks later, when macroscopic tumors had already formed (FIG. 1B, FIG. 8A). Because the SB-Cre^{ERT2} plasmid was co-injected with the N^{ICD} and HA-Akt plasmids, this caused recombination of the Yap and Taz floxed alleles specifically in tumor cells. Deletion of Yap and Taz in tumor cells strongly reduced tumor burden (FIG. 1C-F). Three weeks after Yap/Taz deletion, no macroscopic tumors were visible and gross liver morphology and appearance were relatively normal (FIG. 1C). Histological analysis of mutant livers showed that the liver parenchyma was largely composed of normal hepatocytes and contained only a few remnants of tumors (FIG. 1C). Quantification of the relative tumor area on liver sections (excluding the tumor luminal spaces as these are not true tumor mass) and determination of the absolute tumor mass (by multiplying the relative tumor area with the liver weight) confirmed the macroscopic evaluation and revealed a dramatic tumor reduction upon Yap/Taz deletion (FIG. 1E,F). As controls, we tested the effects of tamoxifen administration on tumor growth by treating wild-type (C57BI/6J) mice that had N-Akt tumors expressing SB-Cre^{ERT2} (FIG. 8B-E), and Yap^{fl/fl};Taz^{fl/fl} double floxed mice with N-Akt tumors that did not express SB-Cre^{ERT2} (FIG. 1C top). Tumor development and growth was not affected in either of these control cohorts compared to tumors growing in non-treated wt animals (FIG. 8B-E). Thus, YAP/TAZ are required for the survival of N-Akt tumor cells.

To mimic systemic YAP/TAZ inhibition we deleted Yap and Taz both in tumor cells and surrounding hepatocytes. We triggered Yap/Taz deletion in hepatocytes by injecting adeno-associated viruses that express Cre (hereafter AAV-Cre) in SB-Cre^{ERT2} Yap/Taz floxed mice. Notably, these AAV-Cre viruses cause Cre expression specifically in hepatocytes but not cholangiocytes or cholangiocarcinoma cells because AAV-Cre was serotype 8, which in the liver only infects hepatocytes, and because Cre expression is under the hepatocyte-specific TBG promoter (FIG. 8F) (Fan et al. 2012; Wang et al. 2010; Yanger et al. 2013). Pilot experiments determined the optimal dosage of the AAV-Cre viruses necessary to promote recombination in virtually every hepatocyte (FIG. 8F) and also showed that AAV infection did not affect tumor development (FIG. 8B-E). Monitoring Cre activity with the Rosa26-LoxP-STOP-LoxP-tdTomato reporter revealed ubiquitous tdTomato expression in virtually all tumor cells and hepatocytes (FIG. 1I). Four weeks after injecting the N^{ICD}, HA-Akt, and SB-Cre^{ERT2} plasmids into Yap^{fl/fl};Taz^{fl/fl} mice, we simultaneously deleted Yap and Taz in N-Akt tumor cells and surrounding hepatocytes by administering tamoxifen and AAV-Cre (FIG. 1B). Strikingly, these mice developed N-Akt tumors that grew to the same size as those in the control mice (FIG. 1C). This was further established by the quantification of relative tumor area and absolute tumor load (FIG. 1D-F). Efficient reduction of YAP/TAZ protein in both tumor cells and normal hepatocytes was confirmed by immunohistochemistry and western blot (FIG. 1G,H). These data show that N-Akt tumor cells require YAP/TAZ for survival when surrounded by wild-type, but not Yap/Taz mutant, hepatocytes.

### 2.2. YAP is Activated in Peritumoral Hepatocytes

The above data indicated that YAP/TAZ do not only function in tumor cells but also in peritumoral hepatocytes. Consistently, elevated levels of YAP, but not TAZ, were detected in hepatocytes around N-Akt tumors, (FIG. 1A, FIG. 2A). In contrast, YAP (or TAZ) levels were barely detected in hepatocytes of normal livers. Note that, as previously shown, YAP was also readily detected in bile ducts and endothelial cells (FIG. 1A) (Wang et al. 2017; Zhang et al. 2010). Consistent with these mouse data, significant YAP and/or TAZ nuclear accumulation was observed in peritumoral hepatocytes of a substantial fraction of human hepatocellular carcinoma (HCC, 44 out of 82) and ICC (13 out of 26) tumors, but not in hepatocytes of healthy human livers (FIG. 2B, FIG. 9A-D).

Accumulation of YAP/TAZ around N-Akt tumors was not due to elevated levels of Yap and Taz mRNA in hepatocytes (FIG. 2C), hence we assayed posttranscriptional regulation by measuring changes in the localization of YAP. However, because YAP is not readily detected in normal hepatocytes and to avoid effects on YAP translation, we assayed ectopically expressed HA-tagged YAP to measure effects on YAP localization. We transfected peritumoral hepatocytes in vivo with sleeping beauty plasmids that expressed HA-tagged YAP under the constitutive Ef1α promoter by hydrodynamic injection of mice that had N-Akt tumors. HA-YAP localization was then visualized and quantified three days later. In normal livers from control mice, HA-YAP was mainly distributed equally in the cytoplasm and the nucleus. In contrast, HA-YAP accumulated in the nuclei of hepatocytes of mice with liver tumors (FIG. 2D-E). This effect was comparable to when YAP was co-expressed with its nuclear partner TEAD4 (FIG. 2D,E). Thus, the presence of liver tumors triggers the nuclear accumulation of YAP in peritumoral hepatocytes.

We next tested for effects on gene expression by transcriptome profiling of FACS purified hepatocytes from normal livers and livers with N-Akt tumors (FIG. 9E,F). RNAseq analysis identified 3273 genes that were significantly upregulated (log 2FC>1, FDR<0.05) and 523 genes that were downregulated (log 2FC<1, FDR<0.05) in peritumoral hepatocytes from N-Akt mice, compared to normal hepatocytes. The upregulated genes were enriched for factors functioning in cell proliferation, stress response, wound healing, angiogenesis, and cell death (FIG. 9G). Notably, gene set enrichment analysis (GSEA) with previously established YAP signatures from YAP-driven hepatocellular carcinoma and from YAP overexpressing MCF10A cells (Sohn et al. 2016; Zhao et al. 2008) detected a prominent Hippo pathway gene expression signature in peritumoral hepatocytes (log 2FC<1, FDR<0.05) (FIG. 2F,G). Among the upregulated genes were prominent YAP targets including Ctgf, Cyr61, Pdgfrβ, Fbn1, Ankrd1, and Birc5. Quantitative RT-qPCR confirmed upregulation of YAP target genes (FIG. 2H). Consistent with YAP activation, hepatocytes ectopically proliferated in tumor-bearing livers. About 6% of peritumoral hepatocytes (marked by HNF4a (HNF4a) expression) as well as other HNF4a negative parenchymal cells expressed Ki67 in addition to the highly proliferating tumor cells (FIG. 9H,I). In normal livers less than 0.2% of hepatocytes expressed Ki67 (FIG. 9H,I). Altogether, these data show that peritumoral hepatocytes ectopically activate YAP and are more proliferative.

### 2.3. Peritumoral YAP Activity Constrains Tumor Growth

To determine the function of YAP in peritumoral hepatocytes, we specifically deleted Yap and Taz in peritumoral hepatocytes, but not in tumor cells, by taking advantage of the fact that AAV-Cre specifically infects hepatocytes but not cholangiocytes or cholangiocarcinoma cells (Fan et al. 2012; Wang et al. 2010; Yanger et al. 2013). For the experiment here, we confirmed the specificity of AAV-Cre for peritumoral hepatocytes by injecting it into R26-LoxP-STOP-LoxP-tdTomato reporter mice with established N-Akt tumors, after 4 weeks of tumor development. Three days later, recombination and activation of the tdTomato reporter was observed in peritumoral hepatocytes but in less than 0.05% of HA-tag positive cholangiocarcinoma cells (FIG. 3A,B).

We next deleted Yap and Taz specifically in peritumoral hepatocytes. We induced cholangiocarcinoma formation in Yap^{fl/fl};Taz^{fl/fl} double floxed mice by hydrodynamic tail vein injection of the N^{ICD} and HA-Akt plasmids (FIG. 3C). After 4 weeks, we deleted Yap and Taz from hepatocytes by injecting these mice with AAV-Cre. For non-deleted controls we injected AAV-Cre into C57BL/6 wt mice with N-Akt tumors or vehicle (PBS) into Yap^{fl/fl};Taz^{fl/fl} double floxed mice with N-Akt tumors. Mice were then analyzed three weeks later, bringing the total length of tumor development to 7 weeks (FIG. 3C). Analysis of Yap and Taz mRNA and protein levels confirmed the deletion of Yap/Taz in peritumoral hepatocytes (FIG. 3D,E).

The deletion of Yap and Taz in peritumoral hepatocytes resulted in increased tumor burden (FIG. 3F-l). We controlled for potential effects of AAV-Cre infection and found that AAV-Cre injection did not affect the total tumor volume in C57BL/6 mice in comparison with vehicle injected C57BL/6 mice (FIG. 8B-E). Therefore, the increased tumor load in Yap/Taz mutant livers is due to decreased YAP and TAZ activity in peritumoral hepatocytes and not to an indirect effect caused by AAV-Cre infection. Strikingly, deletion of Yap and Taz in peritumoral hepatocytes caused an increase in tumor cell proliferation, as evidenced by the increased expression of the proliferation marked Ki67 (FIG. 3J-K). These fast-growing tumors eventually replaced the liver parenchyma as evidenced by increased tumor load while liver to body weight ratio remained the same. Three weeks after peritumoral Yap/Taz deletion only a small percentage of the liver parenchyma remained and much of the tissue was occupied by tumors (40%). In Yap+/Taz+ livers, tumors occupied only 20% of the liver area (FIG. 3H). YAP/TAZ therefore promote the survival of peritumoral hepatocytes, which in turn, suppress the proliferation of the neighboring tumor cells. These data highlight an unexpected activity of YAP/TAZ in peritumoral hepatocytes, which non-autonomously restrains tumor growth.

### 2.4. Lats1/2 Deletion in Peritumoral Hepatocytes Induces Tumor Regression

Our finding that endogenous activation of YAP/TAZ in peritumoral hepatocytes restrains tumor growth prompted us to test whether stronger activation of YAP/TAZ in peritumoral hepatocytes could further increase tumor suppression. To this end, we constitutively activated YAP/TAZ in hepatocytes by conditional deletion of Lats1 and Lats2. To simultaneously delete Lats1 and Lats2 specifically in peritumoral hepatocytes, we injected AAV-Cre into tumor bearing mice that were double homozygous for floxed alleles of Lats1 and Lats2 (Lats1^{fl/fl}, Lats2^{fl/fl})(FIG. 4A). As control mice, we used Lats1^{fl/fl}, Lats2^{fl/fl} mice that were injected with vehicle (PBS). We thus injected AAV-Cre four weeks after N-Akt tumor induction, a time point when livers already had macroscopic tumors (FIG. 4B). Peritumoral deletion of Lats1/2 in mice with N-Akt tumors indeed caused a decrease in inactive phospho-5112-YAP (i.e. increase in active YAP) and an increase in total TAZ levels (FIG. 4C) and resulted in a strong increase in hepatocyte proliferation and liver size (FIG. 4D-F). Thus, deletion of Lats1/2 hyperactivates YAP/TAZ above the levels in wild-type peritumoral hepatocytes.

Strikingly, two weeks after Lats1/2 deletion, most mutant livers showed dramatically reduced tumor load compared to the controls (FIG. 4G-J). Quantifying hepatocyte and tumor areas in liver sections determined that in control mice, liver tumors occupied 40% of the liver area while in Lats1/2 mutant livers tumors occupied less than 5% (FIG. 4H). This dramatic reduction was also reflected in the absolute tumor load where Lats1/2 deletion caused over 70% reduction in tumor load (FIG. 4I). In addition, tumor appearance was altered such that tumors in mutant livers commonly lost their papillary morphology and smaller tumors often appeared to be remnants of regressing tumors (FIG. 4G). Importantly, over half of the mutant livers had reduced tumor load compared to wild-type, and 20% of mutant livers were nearly tumor free (FIG. 4I). These results show that Lats1/2 deletion in peritumoral hepatocytes causes tumor regression.

As Lats1/2 deletion causes strong activation of YAP/TAZ we wanted to test directly whether tumor elimination caused by Lats1/2 deletion was due to hyperactivation of YAP/TAZ or to YAP/TAZ independent functions of LATS1/2. To test this, we simultaneously deleted Yap, Taz, Lats1, and Lats2 in peritumoral hepatocytes. We induced cholangiocarcinoma formation in Yap^{fl/fl};Taz^{fl/fl};Lats1^{fl/fl};Lats2^{fl/fl} quadruple floxed mice by hydrodynamic tail vein injection of the N¹ and HA-Akt plasmids, injected AAV-Cre after 4 weeks of tumor development, and analyzed the mice two weeks later (FIG. 4A). The deletion of Yap/Taz fully rescued the phenotypes on liver growth and tumor suppression caused by Lats1/2 deletion (FIG. 4G). Quadruple mutant livers had a tumor load similar to wild-type livers and no ectopic hepatocyte proliferation and liver overgrowth (FIG. 4D-l). This indicates that Lats1/2 deletion in peritumoral hepatocytes causes cholangiocarcinoma suppression by driving YAP/TAZ activation in peritumoral hepatocytes.

### 2.5. Peritumoral YAP Activation is Sufficient to Trigger Tumor Regression

We next tested whether overexpression of a constitutively active form of YAP (YAP^{1SA}; this is the YAP variant carrying the mutation Ser127Ala; see description hereinabove), due to mutation of the main Lats1/2 phosphorylation site, is sufficient to recapitulate the tumor regression caused by Lats1/2 deletion. We used transgenic mice that conditionally overexpressed human YAP^{1SA} in hepatocytes under the control of a doxycycline (Dox) inducible promoter (TetON system) and where the reverse tetracycline transactivator (rtTA) is expressed under the hepatocyte-specific ApoE promoter (hereafter Apo>hYAP^{1SA})(Dong et al., 2007). Upon doxycycline feeding, Apo>hYAP^{1SA} mice induced hepatocyte proliferation and developed liver overgrowth as previously reported (FIG. 10A,B) (Dong et al. 2007). We then induced N-Akt tumors in Apo>hYAP^{1SA} mice and induced hYAP^{1SA} expression after 4 weeks of tumor development by adding doxycycline to their drinking water (FIG. 5A). As expected, YAP protein levels increased in doxycycline treated Apo>hYAP^{1SA} mice but not in non-treated Apo>hYAP^{1SA} mice or in doxycycline treated C57BL/6 mice (FIG. 10C). Since rtTA was expressed from the hepatocyte-specific ApoE promoter, hYAP^{1SA} was expressed only in peritumoral hepatocytes but not in cholangiocarcinoma cells (FIG. 5B).

After 2 weeks of doxycycline treatment, overexpression of hYAP^{1SA} resulted in a prominent reduction in tumor load compared to non-treated Apo>hYAP^{1SA} siblings (FIG. 5C-E), similar to the one observed in Lats1/2 mutant livers (FIG. 4). Tumor loads of treated Apo>hYAP^{1SA} mice were on average over five times reduced and 5/10 mice had only a few small tumor remnants (FIG. 5C,E). Measuring tumor loads over time showed that non-treated Apo>hYAP^{1SA} control mice had rampant tumor growth over time but treated Apo>hYAP^{1SA} mice had declining tumor loads after hYAP^{1SA} induction (FIG. 5F). Remarkably, two weeks of doxycycline administration was sufficient to extend the survival of Apo>hYAP^{1SA} mice up to 14 weeks after tumor initiation (FIG. 5G). Thus, while half of the control mice died within 6 weeks of tumor initiation, more than 80% of the treated Apo>hYAP^{1SA} mice were still alive by then and survived on average 4 weeks longer than the control mice. However, tumor elimination was not complete and these mice eventually died from re-growing cholangiocarcinoma. These results demonstrate that YAP activation in peritumoral hepatocytes is sufficient to induce tumor regression, and that short periods of YAP activation can prolong the survival of mice with cholangiocarcinoma.

### 2.6. Tumor Cells are Eliminated by Programmed Cell Death

In order to investigate the mechanisms of the tumor cell elimination we first analyzed cell death. N-Akt tumors surrounded by Lats1/2 mutant hepatocytes had drastically elevated numbers of cells that were positive for TUNEL staining (FIG. 6A,B), which assays DNA fragmentation and labels cells undergoing programmed cell death (Elmore 2007; Kressel & Groscurth 1994). Six days after AAV-Cre administration, over 40% of tumor cells were TUNEL positive when surrounded by Lats1/2 mutant hepatocytes, while less than 6% of tumor cells were TUNEL positive when surrounded by wild-type hepatocytes in control livers (FIG. 6B). In particular, control livers exhibited TUNEL positive cells mainly in the center of large tumors, but not in small tumors, while in Lats1/2 mutants small and large tumors were highly positive for TUNEL (FIG. 6A). Thus, tumor cells undergo elevated levels of cell death when surrounded by Lats1/2 mutant hepatocytes.

We monitored immune cell infiltration (CD45 and CD3) and levels of tumor hypoxia (Hif2, Glut1 expression), but we did not detect significant differences in these processes (FIG. 6C,E; FIG. 11). Western blot analysis of regressing tumors from mutant Lats1/2 livers also showed no obvious differences in the amounts of TAZ, YAP, and phosphorylated YAP compared to control tumors (FIG. 6D), eliminating the possibility that the regression phenotype was due to some feedback inhibition on the Hippo pathway itself. Next, we analyzed the contribution of different cell death pathways to the elimination of tumor cells surrounded by Lats1/2 mutant hepatocytes. Regressing tumors showed no difference in the amount of phosphorylated and non-phosphorylated MIkI and Ripk3 compared to control tumors (FIG. 6D, E), while normal livers treated with CCl₄ to provoke acute liver injury showed an increase of pMIkl and decreased levels of Ripk3. Altogether, these results thus suggest that Caspase3 mediated apoptosis and Ripk3 mediated necroptosis might not play significant roles in the elimination of tumor cells. However, regressing tumors of Lats1/2 mutants showed a reduction in the levels of the anti-apoptotic protein Bcl2 when compared to tumors from control mice (FIG. 6E). To determine the contribution of Bcl2 regulation to the elimination of tumor cells, we conditionally overexpressed Bcl2 in tumor cells, starting at the time of Lats1/2 deletion in surrounding hepatocytes. We thus injected the N^{ICD} and HA-Akt plasmids together with a plasmid that conditionally over-expressed Bcl2 under the control of the doxycycline inducible TetON system into Lats1/2 double floxed mice (FIG. 6F). After 4 weeks of tumor development, we injected AAV-Cre to delete Lats1/2 in peritumoral hepatocytes and provided doxycycline in the drinking water to induce Bcl2 expression in tumor cells (FIG. 6G). Strikingly, Bcl2 expression fully rescued the tumor elimination after Lats1/2 deletion (FIG. 6G-J). Bcl2 overexpression in tumors of control mice in which Lats1/2 was not deleted in peritumoral hepatocytes, did not significantly change the size of N-Akt tumors (FIG. 6G-J). The same effect was observed when Bcl2 was overexpressed in tumors of Apo>hYAP^{1SA} mice (FIG. 11C-F). These results show that YAP/TAZ activation in peritumoral hepatocytes triggers programmed cell death in tumor cells, which is prevented by Bcl2 overexpression.

### 2.7. YAP Activation Eliminates Hepatocellular Carcinoma

The above data show that peritumoral hepatocytes suppress the growth of N-Akt induced ICC. We next tested whether this tumor suppressor mechanism is a general mechanism that impacts the growth and development of other liver tumors, in particular hepatocellular carcinoma. We thus tested the effects of peritumoral YAP activation on mouse hepatocellular carcinoma induced by co-expressing Myc and NRas^{G12V} (hereafter Myc-Ras tumors, Seehawer et al. 2018). However, we could not simply use Apo>hYAP^{1SA} or AAV-Cre mediated Lats1/2 deletion to activate YAP in peritumoral hepatocytes because Myc-Ras HCC cells can be infected by AAV-Cre and they also express the ApoE and TBG promoters, which will cause YAP activation in tumor cells. We therefore amended our Apo>hYAP^{1SA} strategy and prevented hYAP^{1SA} expression in tumor cells by co-injecting plasmids expressing shRNAs targeting the rtTA and hYAP^{1SA} transgenes (FIG. 7A). Notably, the YAP^{1SA} transgene expresses human YAP, which allowed us to design shRNAs that specifically target the human Yap^{1SA} transgene but not the endogenous mouse Yap gene. Injection of the rtTA and hYAP^{1SA} targeting shRNA plasmids efficiently inhibited expression of the hYAP^{1SA} transgene in Apo>hYAP^{1SA} mice (FIG. 12E). Thus, combining tumor specific expression of the rtTA and hYAP shRNAs with the Apo>hYAP^{1SA} transgene allowed us to induce hYAP^{1SA} expression specifically in hepatocytes around Myc-Ras HCC.

To test the effects of peritumoral YAPS expression on HCC, we injected the Myc-Ras plasmid together with plasmids expressing shRNAs targeting rtTA, hYAP, or Renillaluciferase as a control into Apo>hYAP^{1SA} mice. Myc-Ras expressing mice had to be euthanized at 6-7 weeks after tail vein DNA injection when they developed large HCC nodules (Seehawer et al. 2018). We thus administered doxycycline 4 weeks after DNA injection to activate hYAP^{1SA} expression. Remarkably, two weeks later, mice expressing the rtTA shRNA or the hYAP shRNA had reduced tumor loads and the combination of both showed nearly complete HCC tumor elimination (FIG. 7C-E, FIG. 12C). In contrast, control mice that expressed the rtTA and hYAP shRNAs but did not receive doxycycline and thus did not induce hYAP^{1SA} expression, had high HCC tumor loads (FIG. 7B-E, FIG. 12). In addition, hYAP^{1SA} expression was not prevented in tumor cells of Apo>hYAP^{1SA} mice expressing the control Renilla luciferase shRNA and these mice had large tumor loads (FIG. 12C). Therefore, YAP activation in peritumoral hepatocytes is sufficient to eliminate Myc-Ras HCC tumors.

### 2.8. YAP Activation Eliminates Metastatic Melanoma in the Liver

Many types of cancer metastasize to the liver (Agarwala et al. 2014; Sherman & Mahvi 2014). Melanoma liver metastases are particularly lethal for patients, especially for those that carry an activating NRAS-mutation (Damsky et al. 2013). To test the ability of YAP-activated hepatocytes to suppress the growth of metastatic melanoma, we injected mouse melanoma cells carrying an activated NRAS mutation and deficient for p16lnk4A by hydrodynamic tail vein injection into Apo>hYAP⁵ mice. The hydrodynamic injection of cells is highly effective in establishing tumor growth in the liver, and to a lesser extend in the kidney and lungs (Li et al. 2011). To avoid immune rejection, we injected melanoma cells derived from a spontaneous tumor isolated from a pure C57BL/6 Tyr:Nras^{Q61K-/+}; Ink4a^{-/-} mouse. Mice injected with 10.000 melanoma cells developed macroscopic tumors after five weeks and had to be euthanized after seven weeks. We administered doxycycline in drinking water three weeks after the cell injection to induce hYAP^{1SA} expression in hepatocytes (FIG. 7F). Remarkably, 2 weeks after YAP activation, Apo>YAP^{1SA} mice showed a very strong reduction in tumor load (98%) (FIG. G-J). Thus, YAP activated hepatocytes promote the spontaneous regression of highly aggressive NRAS-mutant metastatic melanoma cells.

### 2.9. Discussion

Here we describe a novel mechanism of tumor suppression where the normal tissue surrounding liver tumors suppresses tumor growth and survival. In particular, we identified the Hippo pathway effectors YAP/TAZ as critical drivers of this mechanism. The existence of this novel tumor suppressor mechanism is based on two key findings. First, we found that deletion of Yap/Taz in peritumoral hepatocytes of mice with ICC resulted in accelerated tumor growth and increased tumor burden. Second, we found that ectopic hyperactivation of YAP in peritumoral hepatocytes triggered the elimination of well-established models for HCC, ICC, as well as a model for melanoma metastases targeted to the liver. Our data thus identify a mechanism of non-cell autonomous tumor suppression whereby YAP/TAZ action in peritumoral hepatocytes triggers the inhibition of tumor growth.

Notably, we tested models for different primary and metastatic tumors in the liver that represent the three most important tumor types in the liver, namely, HCC, ICC, and liver metastases. We found that all three tumor types were suppressed by peritumoral YAP activation. Thus, the non-autonomous tumor suppressor mechanism that we describe here may be a more general tumor suppressor pathway, at least in the liver. Importantly, our findings may open new avenues for therapeutic approaches to treat different types of liver cancer that still have limited treatment options and poor prognosis. In addition, given that eradicating metastatic disease represents one of the most important clinical challenges faced by modern oncologists, the finding that it can also suppress liver metastases is particularly exciting and may have far-reaching therapeutic implications.

An intriguing aspect of our findings is that the requirement for YAP/TAZ function in tumor cells depends on the level of YAP/TAZ activity in neighboring hepatocytes. Thus, we found that YAP/TAZ were essential for the survival of N-Akt tumor cells when embedded in a wild-type liver but were dispensable for the proliferation and survival of tumor cells when they were surrounded by Yap/Taz mutant hepatocytes. This finding implies that cancer cells of established tumors do not inherently require YAP/TAZ for survival. We want to note, however, that our experiments do not necessarily exclude the existence of an inherent role of YAP/TAZ in tumor cells, such as cell migration and drug resistance. Nonetheless, our data do show that the effect of YAP/TAZ on cell competition is a major function of how they promote the survival and growth of tumor cells in the mouse liver.

Given that YAP/TAZ are heralded as promising targets for cancer therapy, our results have important implications. Our findings suggest that interpretations of previous studies on YAP/TAZ function in cancer would benefit from taking into account the function of YAP/TAZ in tumor surrounding tissues and the possibility that YAP/TAZ act as drivers of cell competition in response to disruption of tissue homeostasis, that is as a consequence of tumor formation. This is especially important for experiments in animals that use tumor specific knockout or hyperactivation of YAP/TAZ. The tumor promoting effect caused by inhibiting YAP/TAZ in the tumor environment also raises questions of potential side effects of therapeutic inhibition of YAP/TAZ as an anti-cancer strategy.

Mechanistically, our Yap/Taz loss-of-function data suggest that the observed tumor suppression reveals the presence of an endogenous mechanism operating in wild-type mice. In healthy livers, YAP/TAZ are not active or required in hepatocytes (Lu et al. 2018). Yet, we found that YAP/TAZ are activated in hepatocytes surrounding growing liver tumors. This was evidenced by nuclear accumulation of YAP in peritumoral hepatocytes of mouse and human liver tumors, by the induction of gene expression profiles that are enriched for YAP signatures, and by cell cycle re-entry of normally quiescent hepatocytes. This YAP/TAZ activation then restrained tumor growth, as peritumoral Yap/Taz deletion accelerated tumor growth. Although it slowed tumor growth, the amount of endogenous YAP/TAZ activation was not sufficient to prevent the development of the N-Akt and Myc-Ras overexpressing tumors. Consistent with this idea, additional hyperactivation of YAP in peritumoral hepatocytes, however, caused regression of these tumors. Thus, the strength of the endogenously activated YAP/TAZ tumor suppression could be amplified by experimental YAP overexpression such that it was then sufficient to prevent the growth of these fast-growing tumors and even caused the near total elimination of some of them. Additionally, it is estimated that millions of cancer cells naturally arise throughout life, yet only few of them develop into tumor masses (Bissell & Hines 2011; Klein 2009). How such originating cancer cells are eliminated remains unknown but the tumor suppressive mechanisms described in this report may be sufficient to eliminate such tumor-initiating cells that may express less powerful oncogenic combinations than those that we used in the ICC and HCC mouse models.

How does this non-autonomous tumor suppressor mechanism work? The observed cellular interactions are reminiscent of cell competition, a process whereby slower growing or otherwise less fit cells (referred to as loser cells) are eliminated from a tissue when they are confronted with cells that grow faster or have higher fitness (referred to as winner cells) (Merino et al. 2016; Vincent et al. 2013).

A handful of signaling pathways have been identified in Drosophila that affect the competitiveness (fitness) of a cell, most notably the Myc transcription factor and the Hippo pathway (Merino et al. 2016; Vincent et al. 2013). A characteristic trait of cell competition is that the survival of a cell does not strictly depend on its absolute level of Myc or Yorkie, the Drosophila homolog of YAP/TAZ, but rather on the relative levels compared to neighboring cells. Thus, in imaginal discs, wild-type cells are winners when confronted with Myc or Yorkie deficient cells but are losers when confronted with cells that overexpress Myc or Yorkie, which converts cells into super-competitors (de la Cova et al. 2004; Neto-Silva et al. 2010). Similarly, in our experiments, N-Akt and Myc-Ras tumor cells change from winner to loser cells depending on the levels of YAP in the surrounding hepatocytes. That is, N-Akt and Myc-Ras cancer cells behave as winner cells when surrounded by wild-type hepatocytes but behave as loser cells when YAP is hyperactivated in surrounding hepatocytes. Data reported here indicate that in the mouse liver, tumor cells engage in a competitive interaction with peritumoral hepatocytes whereby YAP/TAZ activity in tumor cells protects them from cell competition and elimination by the surrounding parenchyma.

In Drosophila, tumorous and hyperproliferating cells of various genotypes and in different tissues often activate Yki and outcompete neighboring wild-type cells (Merino et al. 2016; Vincent et al. 2013). Conversely, stimulating Yki activity and enhancing the survival and competitiveness of stem cells in the Drosophila intestine could suppress the overgrowth of apc mutant stem cells (Suijkerbuijk et al. 2016). Thus, Yorkie/YAP/TAZ govern competitive interactions between tumor cells and surrounding normal cells in different Drosophila tissues and in the mouse liver, raising the possibility of a conserved mechanism. However, we currently do not know how YAP-activated hepatocytes cause the elimination of cancer cells, and despite intense searches, also the mechanisms of cell competition that recognize and trigger the death of loser cells are still not fully understood. Based on genetic evidence, it has been hypothesized that they could include mechanical pressure (Levayer et al. 2015; Levayer et al. 2016; Wagstaff et al. 2016), competition for morphogens (Moreno et al. 2002), signaling through Toll-like receptors (Alpar et al. 2018; Meyer et al. 2014), and redox status (Kucinski et al. 2017). Our current analysis was done in the liver and whether analogous tumor suppressive mechanisms operate in other organs is not yet known. However, because cell competition is present in multiple tissues and across phyla, the tumor suppressive mechanism described here may well be operating in other organs.

YAP/TAZ are best known for their growth and tumor promoting power (Harvey et al. 2013; Zanconato et al. 2016). Thus, elevated levels of YAP/TAZ in tumor cells are generally associated with increased cancer aggressiveness and poor survival. How can this tumor-promoting role of YAP/TAZ be reconciled with the here-identified tumor-suppressing role in peritumoral hepatocytes? The model that YAP/TAZ function as promoters of cellular competitiveness can explain this seeming paradox. In this model, YAP/TAZ activity in cancer cells drives tumor growth and cancer aggressiveness because it enhances the competitiveness of the cancer cells that, due to inherent defects in their function, may be exposed to tumor suppressing effects that are imposed by normal neighboring cells. In support of this hypothesis, ectopic activation of the Drosophila YAP homolog Yorkie can rescue abnormal cells from elimination by cell competition (Merino et al. 2016; Vincent et al. 2013). Indeed, ectopic Yki activation is required for the survival of cells with premalignant genetic aberrations, such as defects in apical-basal polarity, and for eventual tumor formation. Ukewise, YAP/TAZ are required for the initiation of, for example, skin, intestine, and lung adenocarcinoma (Azzolin et al. 2014; Debaugnies et al. 2018; Maglic et al. 2018; Mao et al. 2017; Zanconato et al. 2015) Thus, in the absence of YAP activation, pre-tumor cells may be eliminated by cell competition. The model that YAP/TAZ promote cellular competitiveness can also explain the tumor suppressive effect: here, YAP/TAZ (hyper)activation in peritumoral parenchymal cells increases their fitness therewith exerting a stronger tumor suppressive effect on the tumor cells. Thus, the function of YAP/TAZ in both of these scenarios is the same, namely promotion of competitiveness, but the effect is different depending on which cells activate YAP/TAZ.

In summary, our data identify a novel interaction between liver tumors and the surrounding parenchyma, whereby peritumoral hepatocytes sense the presence of a tumor, activate a YAP-dependent genetic program, and in turn suppress tumor growth. This opens new avenues at least for suppressing or inhibiting liver tumor growth.

### REFERENCES

Agarwala et al. 2014, Cancer 120:781-789.
Alpar et al. 2018, Cell 46:706-719.e705.
Azzolin et al. 2014, Cell 158:157-170.
Bissell & Hines 2011, Nat Med 17:320-329.
Casazza et al. 2014, Oncogene 33:1743-1754.
Caveria et al. 2013, Nature 500: 39-44.
Damsky et al. 2013, Oncogene 33: 2413.
de la Cova et al. 2004, Cell 117:107-116.
Debaugnies et al. 2018. EMBO Rep 19.
Dobin et al. 2013, Bioinformatics 29:15-21.
Dong et al. 2007, Cell 130:1120-1133.
Elmore 2007, Toxicologic pathology 35:495-516.
Fan et al. 2012, J Clin Invest 122:2911-2915.
Fellmann et al. 2013, Cell Reports 5:1704-1713.
Gabrilovich & Nagaraj 2009, Nat Rev Immunol 9:162-174.
Harvey et al. 2013, Nat Rev Cancer 13:246-257.
Heallen et al. 2013, Development 140:4683-4690.
Heallen et al. 2011, Science 332:458-461.
Hogan et al. 2009, Nat Cell Biol 11:460-467.
Johnson & Halder 2014, Nat Rev Drug Discov 13:63-79.
Kim et al. 2013, PLoS One 8: e75449.
Klein 2009, Proc Natl Acad Sci USA 106:859-863.
Kon et al. 2017, Nat Cell Biol 19:530-541.
Kressel & Groscurth 1994, Cell Tissue Res 278:549-556.
Kucinski et al. 2017, Nat Commun 8: 136.
Leung & Brugge 2012, Nature 482; 410.
Levayer et al. 2016, Current biology 26:670-677.
Levayer et al. 2015, Nature 524:476-480.
Li et al. 2011, Cancer Biol Ther 12:737-741.
Love et al. 2014, Genome Biology 15:550.
Lu et al. 2018, Exp Mol Med 50:e423.
Madisen et al. 2010, Nat Neurosci 13:133-140.
Maglic et al. 2018, EMBO J 37:e98642.
Mao et al. 2017, Oncotarget 8:110877-110889.
Marti et al. 2015, Hepatology 62:1497-1510.
Martins et al. 2014, Nature 509:465-470.
Meng et al. 2016, Genes Dev 30:1-17.
Merino et al. 2016, Trends Cell Biol 26:776-788.
Meyer et al. 2014, Science 346:1258236.
Mootha et al. 2003, Nat Genet 34:267-273.
Moreno et al. 2002, Nature 416:755.
Neto-Silva et al. 2010, Dev Cell 19:507-520.
Pei et al. 2015, Oncotarget 6:17206-17220.
Quail & Joyce 2013, Nat Med 19:1423-1437.
Saeed et al. 2003, Biotechniques 34:374-378.
Seehawer et al. 2018, Nature 562:69-75.
Sherman & Mahvi 2014, 53—Liver Metastases. In Abeloff's Clinical Oncology (Fifth Edition), J. E. Niederhuber, J. O. Armitage, J. H. Doroshow, M. B. Kastan, and J. E. Tepper, eds. (Philadelphia: Content Repository Onlyl), pp. 778-793.e773.
Shi et al. 2017, Nat Rev Drug Discov 16:35-52.
Sohn et al. 2016, Clin Cancer Res 22:1256-1264.
Subramanian et al. 2005, Proc Natl Acad Sci USA 102:15545-15550.
Suijkerbuijk et al. 2016, Curr Biol 26:428-438.
Ungefroren et al. 2011, Cell Commun Signaling 9:18-18.
Villa Del Campo et al. 2014, Cell Rep 8:1741-1751.
Vincent et al. 2013, Nat Rev Mol Cell Biol 14:581-591.
Wagstaff et al. 2016, Nat Commun 7:11373.
Wang et al. 2010, Mol Ther 18:118-125.
Wang et al. 2017, Dev Cell 42:462-478.e467.
Xin et al. 2013, Proc Natl Acad Sci USA 110:13839-13844.
Xin et al. 2011, Sci Signal 4, ra70.
Yanger et al. 2013, Genes Dev 27:719-724.
Zanconato et al. 2016, Cancer Cell 29:783-803.
Zanconato et al. 2015, Nat Cell Biol 17:1218-1227.
Zhang et al. 2017, Proc Natl Acad Sci USA 114:498-503.
Zhang et al. 2010, Dev Cell 19:27-38.
Zhao et al. 2008, Genes Dev 22:1962-1971.

1. A method of treating or inhibiting cancer, inhibiting progression of tumor growth, and/or treating or inhibiting tumor metastasis in a subject, the method comprising:
administering to the subject an activator of peritumoral expression and/or function of YES-associated protein (YAP) and/or Tafazzin (TAZ.

2. The method according to claim 1, wherein the activator directly or indirectly activates the function and/or expression of YAP and/or TAZ.

3. method according to claim 1, wherein the subject suffers from liver cancer and/or the subject suffers from a liver tumor.

4. The method according to claim 1, wherein the activator is a pharmacologic compound or a gene therapeutic compound.

5. The method according to claim 1, wherein the activator of expression and/or function of YAP and/or TAZ is a nucleic acid capable of activating expression and/or function of YAP and/or TAZ, or is a nucleic acid capable of blocking inactivation of expression and/or function of YAP and/or TAZ.

6. The the method according to claim 5, wherein the activation of expression and/or function of YAP and/or TAZ is transient or inducible, or wherein the blocking of inactivation of expression and/or function of YAP and/or TAZ is transient or inducible.

7. The method according to claim 5, wherein the activator is a nucleic acid capable of driving expression of YAP or of a constitutively active YAP variant; a nucleic acid capable of driving expression of TAZ or of a constitutively active TAZ variant; a nucleic acid capable of driving expression of any combination of YAP, TAZ, a constitutively active YAP variant, or a constitutively active TAZ variant; or any combination of nucleic acids each individually capable of driving expression of YAP, TAZ, a constitutively active YAP variant, or a constitutively active TAZ variant.

8. The method according to claim 7, wherein the activator is further combined on a same or separate nucleic acid with a gene capable of driving expression of a Transcriptional Enhanced Associated Domain (TEAD) transcription factor.

9. The method according to claim 1, wherein the activator of expression and/or function of YAP and/or TAZ is a glucocorticoid, sphingosine-1-phosphate (SIP), dihydro-SIP, lysophosphatidic acid (LPA), or ethacridine.

10. The method according to claim 1, wherein the administration is local to an organ having a cancer or tumor, or is peritumoral, peripheral, or systemic.

11. The method according to claim 1, wherein the activator is administered in conjunction with macrophage colony-stimulating factor 1 (CSF1), beta-catenin, granulocyte colony-stimulating factor (GCSF), a RAGE-inhibitor, or in conjunction with any combination thereof.

12. The method according to claim 1, wherein the activator is administered in conjunction with an attenuator of cell division, an antifibrotic agent, or in conjunction with any combination thereof.

13. The method according to claim 1 wherein the activator is combined in any way with a further anticancer treatment or antitumor agent.

14. The method according to claim 1, wherein the administration occurs prior to surgical resection of remaining tumor or cancer tissue.

15. (canceled)

16. The method according to claim 3 wherein the liver tumor is liver cholangiocarcinoma, hepatocellular carcinoma or a metastatic liver tumor.

17. (canceled)

18. The method according to claim 1, wherein the activator is administered peritumorally.


SEQUENCE LISTING (18 sequences)

SEQ ID NO: 1 - DNA, 97 bp, Artificial Sequence
  pSBbi-puro-sh1rtTA
tgctgttgacagtgagcgacaacagagaaacagtacgaaatagtgaagccacagatgtatttcgtactgtttctctgttggtgcctactgcctcgga

SEQ ID NO: 2 - DNA, 97 bp, Artificial Sequence
  pSBbi-puro-sh2rtTA
tgctgttgacagtgagcgagcccttgacgattttgacttatagtgaagccacagatgtataagtcaaaatcgtcaagggcgtgcctactgcctcgga

SEQ ID NO: 3 - DNA, 97 bp, Artificial Sequence
  pSBbi-puro-sh3rtTA
tgctgttgacagtgagcgccaggagcatcaagtagcaaaatagtgaagccacagatgtattttgctacttgatgctcctgttgcctactgcctcgga

SEQ ID NO: 4 - DNA, 97 bp, Artificial Sequence
  pSBbi-puro-sh1hYAP
tgctgttgacagtgagcgccgacagtcttcttttgagatatagtgaagccacagatgtatatctcaaaagaagactgtcgatgcctactgcctcgga

SEQ ID NO: 5 - DNA, 97 bp, Artificial Sequence
  pSBbi-puro-sh2hYAP
tgctgttgacagtgagcgcacaggtgatactatcaaccaatagtgaagccacagatgtattggttgatagtatcacctgtatgcctactgcctcgga

SEQ ID NO: 6 - DNA, 20 bp, Artificial Sequence
  Primer
gccatgttgttgtctgatcg

SEQ ID NO: 7 - DNA, 20 bp, Artificial Sequence
  Primer
cctgatgatgtaccactgcc

SEQ ID NO: 8 - DNA, 20 bp, Artificial Sequence
  Primer
tgccatgtggtgattttctc

SEQ ID NO: 9 - DNA, 20 bp, Artificial Sequence
  Primer
cctatgacgtgaccgacgag

SEQ ID NO: 10 - DNA, 20 bp, Artificial Sequence
  Primer
gcttggcgattttaggtgtc

SEQ ID NO: 11 - DNA, 20 bp, Artificial Sequence
  Primer
cagactggagaagcagagcc

SEQ ID NO: 12 - DNA, 20 bp, Artificial Sequence
  Primer
tttacagttgggctggaagc

SEQ ID NO: 13 - DNA, 20 bp, Artificial Sequence
  Primer
caccgctctgaaagggatct

SEQ ID NO: 14 - DNA, 21 bp, Artificial Sequence
  Primer
tgaggctgaaccgctataaga

SEQ ID NO: 15 - DNA, 20 bp, Artificial Sequence
  Primer
cagtgcaacaccagatccat

SEQ ID NO: 16 - DNA, 20 bp, Artificial Sequence
  Primer
cgtcccgtagacaaaatggt

SEQ ID NO: 17 - DNA, 20 bp, Artificial Sequence
  Primer
ttgatggcaacaatctccac

SEQ ID NO: 18 - protein, 6 aa, Artificial Sequence
  Consensus amino acid (6 amino acids) motif YAP- phosphorylation
  misc_feature (2)..(2): Xaa can be any naturally occurring amino acid
  MISC_FEATURE (3)..(3): X can be R or H
  misc_feature (4)..(5): Xaa can be any naturally occurring amino acid
HXXXXS